SEQUENCE LISTING (22 sequences)

SEQ ID NO: 1 - DNA, 2589 bp, Mus musculus
  source 1..2589: mol_type = genomic DNA
acattacatgattcttaccagctttggagatgacatgtggcttctaacaactctgctactttgggttccagtcggtggggaagtggttaatgccaccaaggctgtgatcaccttgcagcctccatgggtcagtattttccagaaggaaaatgtcactttatggtgtgaggggcctcacctgcctggagacagttccacacaatggtttatcaacggaacagccgttcagatctccacgcctagttatagcatcccagaggccagttttcaggacagtggcgaatacaggtgtcagataggttcctcaatgccaagtgaccctgtgcagttgcaaatccacaatgattggctgctactccaggcctcccgcagagtcctcacagaaggagaacccctggccttgaggtgtcacggatggaagaataaactggtgtacaatgtggttttctatagaaatggaaaatcctttcagttttcttcagattcggaggtcgccattctgaaaaccaacctgagtcacagcggcatctaccactgctcaggcacgggaagacaccgctacacatctgcaggagtgtccatcacggtgaaagagctgtttaccacgccagtgctgagagcatccgtgtcatctcccttcccggaggggagtctggtcaccctgaactgtgagacgaatttgctcctgcagagacccggcttacagcttcacttctccttctacgtgggcagcaagatcctggagtacaggaacacatcctcagagtaccatatagcaagggcggaaagagaagatgctggattctactggtgtgaggtagccacggaggacagcagtgtccttaagcgcagccctgagttggagctccaagtgcttggtccccagtcatcagctcctgtctggtttcacatcctgttttatctgtcagtgggaataatgttttcgttgaacacggttctctatgtgaaaatacacaggctgcagagagagaagaaatacaacttagaagtccctttggtttctgagcagggaaagaaagcaaattcctttcagcaagttagaagcgatggcgtgtatgaagaagtaacagccactgcgagccagaccacaccaaaagaagcgcccgatggacctcgaagctcagtgggtgactgtggacccgagcagcctgaaccccttcctcccagtgacagtactggggcacaaacttcccaaagttgaccctgaaactgtgggaccatggcatgcaactcttaaataaagcaaatatacagactggatccggctgagacaagctgggtaatcagacatttgaaaggagacctataccaaagggatcttgcaacacatggagtcaggtcacagcgggggttgtcgaatgtttgaccttatggagcagggaaacaggaagtgaatcccacaggactccccccccccgcccatccccctccaggccgccccggacaggacccagctctggaagactccagtctgagacttgcggaaccagagcaggggtgagattcctgcccagaagggacagctgtgccatcccctcacagggtggatgggttcagggaaaggcctccccagggacggcctgcgtgtcaggggagcagacgctgatacagacagctccatagcctgggctaaagctggctaagacccggtggtcatcctgagagcatcggaatttgtgctctccttcctaccgtctctcttcatgcaccctccccagatttgctgcccacgaccctcaaaggacatagtggcggcagctaaagagtgaagtgtcagcagtaatccatccatctaacctccctcaggtccagatacccccacccccaaactcccacactctaggggccttttcaggcagcctgcatgtggtgtcttagcagagctatggtacaaaggcttttagctctatcattatctgacaagcagacagcaccctcaggtgctctcattgggtggtgagagctttctccagcctgtaccacctgtaagctggagtgtggggcgggaacactggcccaaagcgtccctattggaaggcacggcttacatgggtgtcacaaatgcccttagaccacgcaggaagaccgaattctagaaacaaggagtagatcatgtctccacttactgtcactcctaaggatcccctgaaggtcttggagcttcacatccctggaactctagggtctgccgtgctagaggtcccagtctgcagagtgggtgtggcatagcctgagcctccctggatgtgaacattagcaaggtatattgggacctttataaccagggaccaataggcatgagagggaccgggataatggaccacagtcacaggaggagatacactctgttgtacaatgcatgcagaaactgtcaaaaacagtgtgggagctggagagatgatcaggggttaagaacacttcctgctcttccagaggacctgagttcactttttgtaactgcttgtaagtccagatgtcgtcttctgatctctttcaagcacccacatgtgcagggcatgcagacacagacatatgaacaagaacaattaaaaaataaattataactgc

SEQ ID NO: 2 - DNA, 20 bp, synthetic construct
  source 1..20: mol_type = other DNA
tctgggtaccgaaaggcggg

SEQ ID NO: 3 - DNA, 20 bp, synthetic construct
  source 1..20: mol_type = other DNA
tcccaataaaacttcaccag

SEQ ID NO: 4 - DNA, 20 bp, synthetic construct
  source 1..20: mol_type = other DNA
ggatccggctgagacaagct

SEQ ID NO: 5 - DNA, 20 bp, synthetic construct
  source 1..20: mol_type = other DNA
atggagtcaggtcacagcgg

SEQ ID NO: 6 - DNA, 20 bp, synthetic construct
  source 1..20: mol_type = other DNA
atggagtcaggtcacagcgg

SEQ ID NO: 7 - DNA, 20 bp, synthetic construct
  source 1..20: mol_type = other DNA
gcctgagtctcagtttccaa

SEQ ID NO: 8 - DNA, 22 bp, synthetic construct
  source 1..22: mol_type = other DNA
caggctatggagctgtctgtat

SEQ ID NO: 9 - DNA, 20 bp, synthetic construct
  source 1..20: mol_type = other DNA
cgtctcagactggagtcttc

SEQ ID NO: 10 - DNA, 735 bp, Mus musculus
  source 1..735: mol_type = genomic DNA
tcacaggttgagaaacactggcctagtgagattagaaaagcacagaattttagaaaaccagaggcagccctattaggctggattctcagtgcccccaaatctgtccctttcataagaaagcagacaggtttttctcctcaggagctaggaaggcttcaagggcctgagtctcagtttccaatggagaagtggatggagatgctttaccctgcccccagcccagaggcagctagcgctgagtggagggaaactggcttgaggcggtttgctgtggtttgagacccacttcattaactctggtgcttctttctctggcaaccaaaccctaccatctggatccaaatcaaacaacctagaaaagctcccaggacagtctgggtaccgaaaggcgggcggctagcttcctgggttctcacttgctaatcttttaaaactaatgtgaaaatacattgcccttttgtgctcccaataaaacttcaccagtggctcaggttctgcctttaactcactcaggggtttcatcgtcgttgtcgtttgtttgtttgtttgtttgtttttctctcttttctacagttaatgccaccaaggctgtgatcaccttgcagcctccatgggccagtattttccagaaggaaaatgtcactttatggtgtgaggggcctcacctgcctggagacagttccacacaatggtttatcaatggaacagtcgttcagacctccacacctagttata

SEQ ID NO: 11 - DNA, 735 bp, Mus musculus
  source 1..735: mol_type = genomic DNA
tataactaggtgtggaggtctgaacgactgttccattgataaaccattgtgtggaactgtctccaggcaggtgaggcccctcacaccataaagtgacattttccttctggaaaatactggcccatggaggctgcaaggtgatcacagccttggtggcattaactgtagaaaagagagaaaaacaaacaaacaaacaaacaaacgacaacgacgatgaaacccctgagtgagttaaaggcagaacctgagccactggtgaagttttattgggagcacaaaagggcaatgtattttcacattagttttaaaagattagcaagtgagaacccaggaagctagccgcccgcctttcggtacccagactgtcctgggagcttttctaggttgtttgatttggatccagatggtagggtttggttgccagagaaagaagcaccagagttaatgaagtgggtctcaaaccacagcaaaccgcctcaagccagtttccctccactcagcgctagctgcctctgggctgggggcagggtaaagcatctccatccacttctccattggaaactgagactcaggcccttgaagccttcctagctcctgaggagaaaaacctgtctgctttcttatgaaagggacagatttgggggcactgagaatccagcctaatagggctgcctctggttttctaaaattctgtgcttttctaatctcactaggccagtgtttctcaacctgtga

SEQ ID NO: 12 - DNA, 20 bp, synthetic construct
  source 1..20: mol_type = other DNA
tctgggtaccgaaaggcggg

SEQ ID NO: 13 - DNA, 20 bp, synthetic construct
  source 1..20: mol_type = other DNA
tcccaataaaacttcaccag

SEQ ID NO: 14 - protein, 54 aa, Mus musculus
VNATKAVITLQPPWASIFQKENVTLWCEGPHLPGDSSTQWFINGTVVQTSTPSY

SEQ ID NO: 15 - DNA, 588 bp, Mus musculus
  source 1..588: mol_type = genomic DNA
gtaacagccactgcgagccagaccacaccaaaagaagcgcccgacggacctcgaagctcagtgggtgactgtggacccaagcagcctgaaccccttccttccagtgacagtaccagggcacaaacttcccaaagttgaccctgaaattgtgggaccatggcatgcaactcttaaataaagcaaatatacagactggatccggctgagacaagctaggtaatcagacatttgaaaggagacctataccaaagggatcttgcaacacatggagtcaggtcacagcgggggttgtcgaatgtttgaccttatggagtagggaaacaggaagtgaatcccgcaggactccccccccccgcccatccccctccaggcctccccagacaggacccagctctggaagactccagtctgagacgcacggaaccagagcaggggtgagattcctgcccagaagggacagctgtgccatcccctcacagggtggatgggttcagggaaaggcctccccagggacggcctgtgtgtcaggggagcagacgctgatacagacagctccatagcctgggctaaagctggctaagacccggt

SEQ ID NO: 16 - DNA, 588 bp, Mus musculus
  source 1..588: mol_type = genomic DNA
accgggtcttagccagctttagcccaggctatggagctgtctgtatcagcgtctgctcccctgacacacaggccgtccctggggaggcctttccctgaacccatccaccctgtgaggggatggcacagctgtcccttctgggcaggaatctcacccctgctctggttccgtgcgtctcagactggagtcttccagagctgggtcctgtctggggaggcctggagggggatgggcggggggggggagtcctgcgggattcacttcctgtttccctactccataaggtcaaacattcgacaacccccgctgtgacctgactccatgtgttgcaagatccctttggtataggtctcctttcaaatgtctgattacctagcttgtctcagccggatccagtctgtatatttgctttatttaagagttgcatgccatggtcccacaatttcagggtcaactttgggaagtttgtgccctggtactgtcactggaaggaaggggttcaggctgcttgggtccacagtcacccactgagcttcgaggtccgtcgggcgcttcttttggtgtggtctggctcgcagtggctgttac

SEQ ID NO: 17 - protein, 44 aa, Mus musculus
QPLRARPHQKKRPTDLEAQWVTVDPSSLNPFLPVTVPGHKLPKV

SEQ ID NO: 18 - DNA, 20 bp, synthetic construct
  source 1..20: mol_type = other DNA
ggatccggctgagacaagct

SEQ ID NO: 19 - DNA, 20 bp, synthetic construct
  source 1..20: mol_type = other DNA
atggagtcaggtcacagcgg

SEQ ID NO: 20 - DNA, 133 bp, Mus musculus
  source 1..133: mol_type = genomic DNA
atgattcttaccagctttggagatgacatgtggcttctaacaactctgctactttgggttccagtcggtggggaagtggttgtgggaccatggcatgcaactcttaaataaagcaaatatacagactggatcc

SEQ ID NO: 21 - DNA, 133 bp, Mus musculus
  source 1..133: mol_type = genomic DNA
ggatccagtctgtatatttgctttatttaagagttgcatgccatggtcccacaaccacttccccaccgactggaacccaaagtagcagagttgttagaagccacatgtcatctccaaagctggtaagaatcat

SEQ ID NO: 22 - protein, 26 aa, Mus musculus
MILTSFGDDMWLLTTLLLWVPVGGEV

(19) United States
(12) Patent Application Publication
(10) Pub. No.: US 2025/0212854 A1
(43) Pub. Date: Jul. 3, 2025

(54) IMMUNODEFICIENT FCGR1 KNOCKOUT MOUSE MODELS

(71) Applicant: The Jackson Laboratory, Bar Harbor, ME (US)

(72) Inventor: Leonard D. Shultz, Bar Harbor, ME (US)

(73) Assignee: The Jackson Laboratory, Bar Harbor, ME (US)

(21) Appl. No.: 18/850,400

(22) PCT Filed: Mar. 24, 2023
(86) PCT No.: PCT/US2023/064918

§ 371 Date: Sep. 24, 2024

Related U.S. Application Data
(60) Provisional application No. 63/323,773, filed on Mar. 25, 2022.

(51) Int. Cl. A01K 67/0276; A01K 67/0271; A01K 67/0278; C07K 14/54; C07K 14/715; C07K 14/735

(52) U.S. Cl. CPC A01K 67/0276; A01K 67/0271; A01K 67/0278; C07K 14/5443; C07K 14/70535; C07K 14/7155; A01K 2207/12; A01K 2217/075; A01K 2227/105; A01K 2267/03

(57) ABSTRACT

The present disclosure provides, in some aspects, an immunodeficient NOD-Fcgr1^{null} mouse that may be used to measure human IgG antibody pharmacokinetics and activity, produce human IgG antibodies, and model human disease treatment.

### RELATED APPLICATIONS

This application claims the benefit under 35 U.S.C. § 119(e) of U.S. provisional application No. 63/323,773, filed Mar. 25, 2022, which is incorporated by reference herein in its entirety.

### GOVERNMENT LICENSE RIGHTS

This invention was made with government support under AI132963 awarded by National Institutes of Health. The government has certain rights in the invention.

### REFERENCE TO AN ELECTRONIC SEQUENCE LISTING

The contents of the electronic sequence listing (J022770112WO00-SEQ-EMB.xml; Size: 25,766 bytes; and Date of Creation: Mar. 23, 2023) are herein incorporated by reference in its entirety.

### BACKGROUND

Fragment crystallizable (Fc) receptors are cell surface proteins that bind immunoglobulin (Ig) antibodies and internalize the Ig antibodies into the cell. The internalized Ig antibodies may be recycled to the cell surface to re-enter circulation or trafficked to the lysosome. Ig antibodies that are trafficked to the lysosome are degraded into peptides that are loaded onto major histocompatibility class I (MHC-I) and major histocompatibility class II (MHC-II) proteins to be presented as antigens on the cell surface. These antigens regulate immune cell signaling.

Mice have five types of Fc receptors. These are Fc gamma receptor I (FcγRI), Fc gamma receptor IIB (FcγRIIB), Fc gamma receptor III (FcγRIII), Fc gamma receptor IV (FcγRIV), and Fc receptor neonatal (FcRn) (Bruhns P., Blood, 2012; 119 (24): 5640-5649). FcγRI, FcγRIII, FcγRIV, and FcRn activate immune signaling and FcγRIIB inhibits immune signaling. Dendritic cells (DCs) express FcγRI, FcγRIIB, FcγRIII, and FcγRIV; mouse Ly6C^{lo} monocytes, macrophages, and neutrophils express FcγRIV; B cells express FcγRIIB; and natural killer and natural killer T cells express FcγRIII. During the prenatal period, neutrophils, splenic monocytes, B cells, vascular endothelial cells, and epithelial cells in the intestine express FcRn.

### SUMMARY

Some aspects of the present disclosure provide an immunodeficient mouse comprising a mouse Fc gamma receptor 1 null (Fcgr1^{null}) allele and a mouse interleukin-2 receptor gamma null (IL-2Rγ^{null}) allele.

In some embodiments, the mouse has a non-obese diabetic (NOD) genetic background (e.g., NOD/ShiLtJ).

In some embodiments, the mouse is homozygous for the IL-2Rγ^{null} allele and is homozygous for the Fcgr1^{null} allele.

In some embodiments, the mouse further comprises Prkdc^{scid} allele.

In some embodiments, the mouse is homozygous for the Prkdc^{scid} allele.

In some embodiments, the mouse further comprises a Rag1^{null} allele.

In some embodiments, the mouse is homozygous for the Rag1^{null} allele.

Some aspects of the present disclosure provide an immunodeficient mouse comprising a mouse Fc gamma receptor 1 null (Fcgr1^{null}) allele, wherein the mouse comprises an NOD.Cg-Prkdc^{scid}Il2rg^{tm1Wj1}/SzJ background.

In some embodiments, the mouse lacks mouse T cells, B cells, and/or natural killer (NK) cells.

In some embodiments, macrophage and/or dendritic cell function in the mouse is defective.

In some embodiments, the immunodeficient mouse is engrafted with human hematopoietic stem cells (HSCs).

In some embodiments, the immunodeficient mouse is engrafted with human peripheral blood mononuclear cells (PBMCs).

In some embodiments, the immunodeficient mouse is engrafted with human diseased cells.

In some embodiments, the human diseased cells are human tumor cells.

In some embodiments, the human tumor cells are human cancer cells.

In some embodiments, the mouse Fcgr1^{null} allele comprises a deletion in a region of exons 3-6, relative to an endogenous unmodified mouse Fcgr1 gene. In some embodiments, the mouse Fcgr1^{null} allele comprises a deletion of exons 3-6.

In some embodiments, the immunodeficient mouse further comprises a human IL-15 transgene. In some embodiments, the human IL-15 transgene is integrated into the genome of the mouse.

Other aspects of the present disclosure provide a method comprising administering an antibody to the immunodeficient mouse described herein.

In some embodiments, the antibody is a monoclonal antibody.

In some embodiments, the antibody is a human or humanized antibody.

In some embodiments, the antibody is an IgG1, IgG2, IgG3 or IgG4 antibody.

In some embodiments, the antibody is an immune checkpoint inhibitor (ICI) antibody. In some embodiments, the ICI antibody is selected from anti-PD-1 antibodies, anti-PD-L1 antibodies, and anti-CTLA-4 antibodies.

In some embodiments, the method further comprises assaying a biological sample from the mouse for a therapeutic effect of the antibody.

In some embodiments, the therapeutic effect is decreased growth of human diseased cells, relative to a control.

In some embodiments, the method further comprises assaying a biological sample from the mouse for a circulating level of the antibody.

In some embodiments, the circulating level of the antibody is at least 5-fold, at least 10-fold, at least 15-fold, or at least 20-fold higher, relative to a control.

In some embodiments, the assaying is at least 21, at least 28, or at least 35 days post-administration of the antibody.

Some aspects of the present disclosure provide a method comprising administering human cells to the immunodeficient mouse described herein. In some embodiments, the human cells are HSCs. In other embodiments, the human cells are human PBMCs. In some embodiments, the human cells are human diseased cells. In some embodiments, the human diseased cells are human tumor cells. In some embodiments, the human tumor cells are human cancer cells.

Other aspects of the present disclosure provide a guide RNA comprising the sequence of any one of SEQ ID NOs: 2-5.

Yet other aspects of the present disclosure provide a method of producing an immunodeficient mouse described herein comprising introducing one or more guide RNA and a Cas protein into a mouse embryo (e.g., having any one of the immunodeficient genetic backgrounds described herein); transplanting the mouse embryo into a pseudopregnant female mouse; collecting F1 mice born from the pseudopregnant female; and breeding the F1 mice.

In some embodiments the one or more guide RNA binds to a region upstream from exon 3 and/or downstream from exon 6 of the mouse Fcgr1 gene, optionally a guide RNA comprising the sequence of any one of SEQ ID NOs: 2-5.

In some embodiments, the mouse embryo is an immunodeficient mouse embryo, optionally having an NOD genetic background, and optionally comprising one or more of (a) an IL-2Rγ^{null} allele and (b) an Prkdc^{scid} allele or a Rag1^{null} allele.

### BRIEF DESCRIPTION OF THE DRAWINGS

FIG. 1. Upstream mouse Fcgr1 deletion allele targeting strategy.

FIG. 2. Downstream mouse Fcgr1 deletion allele targeting strategy

FIG. 3. Mouse Fcgr1 deletion allele sequencing strategy.

FIG. 4. Mouse Fcgr1 deletion allele predicted protein translation. Deletion of Fcgr1 exons 3-6 is predicted to encode a Fcgr1 protein with an early truncation after amino acid 26.

FIG. 5. Founder results for NSG-Fcgr1^{null} mice. “Wild” is wild-type, “het” is Fcgr1^{null} heterozygous mice, and “hom” is Fcgr1^{null} homozygous mice. The bolded text represents NSG-Fcgr1^{null} mice heterozygous for just the targeted deletion of exons 3-6 from Fcgr1.

FIG. 6. Founder results for NSG-Fcgr1^{null}Tg(Hu-IL15) mice. “Wild” is wild-type, “het” is Fcgr1^{null} heterozygous mice, and “hom” is Fcgr1^{null} homozygous mice. The bolded text represents live-born NSG-Fcgr1^{null}Tg(Hu-IL15) mice heterozygous for the targeted deletion of exons 3-6 from Fcgr1.

FIG. 7. Increased survival of trastuzumab (Herceptin®) IgG antibody in the circulation of NSG-Fcgr1^{null} mice compared to NSG™ mice.

FIG. 8. Pharmacokinetic analysis of trastuzumab IgG antibody in NSG™ and NSG-Fcgr1^{null} (NSG™ Fcgr1 KO) mice. “SD” is standard deviation, “CV” is variation, “AUC” is area under the curve, “Lambda.z” is the estimated terminal elimination rate constant, “Vss” is the steady state volume of distribution, “CL” is trastuzumab clearance, and “Vd” is the volume of distribution.

FIG. 9. Increased survival of pembrolizumab (Keytruda®) IgG antibody in the circulation of NSG-Fcgr1^{null} mice compared to NSG™ mice FIG. 10. Pharmacokinetic analysis of pembrolizumab IgG antibody in NSG™ and NSG-Fcgr1^{null} (NSG™ Fcgr1 KO) mice.

FIG. 11. Increased levels of human IgG antibodies following engraftment of NSG-Fcgr1^{null} mice with CD34⁺ umbilical cord hematopoietic stem cells (HSCs) compared with NSG™ mice engrafted with CD34⁺ HSCs.

### DETAILED DESCRIPTION

Human immunoglobulins (IgGs) are widely used as therapeutic antibodies. Mouse models for testing the pharmacokinetics and efficacy of new antibodies, however, are limited. This is partly because the human IgG antibodies bind with high affinity to the mouse FcγR1 receptor (encoded by the Fcgr1 gene), resulting in rapid clearance of the antibodies, which in turn greatly reduces the half-life of the antibodies. Human IgGs administered to immunodeficient NSG™ mouse model (i.e., NOD.Cg-Prkdc^{scid} Il2rg^{tm1Wj1}/SzJ), for example, are cleared much more rapidly than those administered to humans. This rapid clearance rate limits the use of the immunodeficient mouse models for assessing therapeutic efficacy of human IgG monoclonal antibody (mAb) drugs against patient-derived xenografts and other engrafted human cells (e.g., tumor cells), for example.

The present disclosure advances the field by providing, in some aspects, an immunodeficient mouse model that does not express FcγR1 (e.g., an immunodeficient Fcgr1 gene knockout, e.g., NSG-Fcgr1^{null}). Production of this genetically engineered mouse model involved a complex set of experimental procedures, including gene editing design and implementation as well as a carefully planned breeding scheme. Specifically, exons 3-6 of the endogenous mouse Fcgr1 gene were edited out (deleted) using CRISPR/Cas9 gene editing in both an NSG™ background strain and in an NSG™ background strain that includes a human interleukin-15 (IL-15) transgene (i.e., NSG-Tg(Hu-IL15)). The NSG-Fcgr1^{null}-Tg(Hu-IL15) mouse model has the added advantage of producing human natural killer (NK) cells to induce antibody-dependent cell-mediated cytotoxicity (ADCC) in response to human IgG.

The data provided herein shows, in some instances, an unexpected ˜20-fold to ˜30-fold increase in the circulating half-life of human IgG antibodies administered to the immunodeficient Fcgr1^{null} mice, relative to a control. Additionally, the immunodeficient Fcgr1^{null} mice develop ˜20-fold higher levels of circulating human IgG antibodies following engraftment with human hematopoietic stem cells (HSCs), relative to a control. Thus, the present disclosure provides the field with immunodeficient mouse models that can be used to assess the pharmacokinetics and efficacy of human monoclonal antibody therapies more accurately, particularly against human cancers and other diseases, for example.

### Immunodeficient Fcgr1^{null} Mouse Models

In some aspects, the present disclosure provides an immunodeficient mouse in which an Fc receptor (FcR) has been knocked out. Thus, the immunodeficient mice provided herein comprise an FcR “null” allele—an allele that encodes a nonfunctional product. Immunoglobulin Fc receptors are membrane molecules expressed by several hematopoietic cell populations that bind the Fc region of an immunoglobulin (Igs). Non-limiting examples of Fc receptors that may be knocked out in an immunodeficient mouse strain include: Fc gamma receptor (FcγR/FcgR), Fc epsilon receptor (FcεR, FceR), Fc alpha receptor (FcαR, FcaR), Fc mu receptor (FcμR, FcmR), Fc receptor neonatal (FcRn), or a combination thereof.

In some embodiments, a Fc gamma receptor is knocked out in an immunodeficient mouse. Non-limiting examples of Fc gamma receptors include those encoded by Fcgr1 (CD64), Fcgr2, Fcgr3 (CD16), Fcgr3a (CD32a), Fcgr3b (CD16b), Fcgr3c (CD16c), and Fcgr4. Fc gamma receptors bind the Fc region of immunoglobulin gamma (IgG) molecules. For example, Fc gamma receptors bind IgG-coated molecules, such as opsonized pathogens and immune complexes. Cross-linking between the Fc gamma receptor and IgG leads to internalization and degradation of the IgG-coated molecule. Thus, in some aspects, the present disclosure provides a mouse with decreased IgG internalization and degradation. Fc gamma receptors bind IgG through the crystallizable (Fc) region and internalize IgG into the cell. Once internalized, both the receptor and IgG are delivered to and degraded by lysosomes. This degradation of the IgG prevents it from being recycled into systemic circulation. Portions of the degraded IgG molecule are then loaded onto major histocompatibility complex class 1 (MHC-I) and major histocompatibility complex class II (MHC-II) molecules for presentation as antigens on the cell surface.

In some embodiments, an Fcgr1 gene (e.g., Ensembl #ENSMUSG00000015947; GenBank NM_010186) is knocked out in an immunodeficient mouse. Such a mouse may be referred to herein as an Fcgr1^{null} mouse. An Fcgr1^{null} mouse does not produce detectable levels of mouse FcγR1 protein (e.g., GenBank NP_034316.1).

(SEQ ID NO: 1)
acattacatg attcttacca gctttggaga tgacatgtgg cttctaacaa ctctgctact ttgggttcca
gtcggtgggg aagtggttaa tgccaccaag gctgtgatca ccttgcagcc tccatgggtc agtattttcc
agaaggaaaa tgtcacttta tggtgtgagg ggcctcacct gcctggagac agttccacac aatggtttat
caacggaaca gccgttcaga tctccacgcc tagttatagc atcccagagg ccagttttca ggacagtggc
gaatacaggt gtcagatagg ttcctcaatg ccaagtgacc ctgtgcagtt gcaaatccac aatgattggc
tgctactcca ggcctcccgc agagtcctca cagaaggaga acccctggcc ttgaggtgtc acggatggaa
gaataaactg gtgtacaatg tggttttcta tagaaatgga aaatcctttc agttttcttc agattcggag
gtcgccattc tgaaaaccaa cctgagtcac agcggcatct accactgctc aggcacggga agacaccgct
acacatctgc aggagtgtcc atcacggtga aagagctgtt taccacgcca gtgctgagag catccgtgtc
atctcccttc ccggagggga gtctggtcac cctgaactgt gagacgaatt tgctcctgca gagacccggc
ttacagcttc acttctcctt ctacgtgggc agcaagatcc tggagtacag gaacacatcc tcagagtacc
atatagcaag ggcggaaaga gaagatgctg gattctactg gtgtgaggta gccacggagg acagcagtgt
ccttaagcgc agccctgagt tggagctcca agtgcttggt ccccagtcat cagctcctgt ctggtttcac
atcctgtttt atctgtcagt gggaataatg ttttcgttga acacggttct ctatgtgaaa atacacaggc
tgcagagaga gaagaaatac aacttagaag tccctttggt ttctgagcag ggaaagaaag caaattcctt
tcagcaagtt agaagcgatg gcgtgtatga agaagtaaca gccactgcga gccagaccac accaaaagaa
gcgcccgatg gacctcgaag ctcagtgggt gactgtggac ccgagcagcc tgaacccctt cctcccagtg
acagtactgg ggcacaaact tcccaaagtt gaccctgaaa ctgtgggacc atggcatgca actcttaaat
aaagcaaata tacagactgg atccggctga gacaagctgg gtaatcagac atttgaaagg agacctatac
caaagggatc ttgcaacaca tggagtcagg tcacagcggg ggttgtcgaa tgtttgacct tatggagcag
ggaaacagga agtgaatccc acaggactcc ccccccccgc ccatccccct ccaggccgcc ccggacagga
cccagctctg gaagactcca gtctgagact tgcggaacca gagcaggggt gagattcctg cccagaaggg
acagctgtgc catcccctca cagggtggat gggttcaggg aaaggcctcc ccagggacgg cctgcgtgtc
aggggagcag acgctgatac agacagctcc atagcctggg ctaaagctgg ctaagacccg gtggtcatcc
tgagagcatc ggaatttgtg ctctccttcc taccgtctct cttcatgcac cctccccaga tttgctgccc
acgaccctca aaggacatag tggcggcagc taaagagtga agtgtcagca gtaatccatc catctaacct
ccctcaggtc cagatacccc cacccccaaa ctcccacact ctaggggcct tttcaggcag cctgcatgtg
gtgtcttagc agagctatgg tacaaaggct tttagctcta tcattatctg acaagcagac agcaccctca
ggtgctctca ttgggtggtg agagctttct ccagcctgta ccacctgtaa gctggagtgt ggggcgggaa
cactggccca aagcgtccct attggaaggc acggcttaca tgggtgtcac aaatgccctt agaccacgca
ggaagaccga attctagaaa caaggagtag atcatgtctc cacttactgt cactcctaag gatcccctga
aggtcttgga gcttcacatc cctggaactc tagggtctgc cgtgctagag gtcccagtct gcagagtggg
tgtggcatag cctgagcctc cctggatgtg aacattagca aggtatattg ggacctttat aaccagggac
caataggcat gagagggacc gggataatgg accacagtca caggaggaga tacactctgt tgtacaatgc
atgcagaaac tgtcaaaaac agtgtgggag ctggagagat gatcaggggt taagaacact tcctgctctt
ccagaggacc tgagttcact ttttgtaact gcttgtaagt ccagatgtcg tcttctgatc tctttcaagc
acccacatgt gcagggcatg cagacacaga catatgaaca agaacaatta aaaaataaat tataactgc

A mouse Fcgr1 gene may be knocked out by deleting or introducing a modification (e.g., mutation) at any location in a gene sequence or a regulatory gene sequence (e.g., promoter, 5′ UTR, enhancer, insulator, 3′ UTR), provided the resulting sequence does not encode a functional protein. In some embodiments, a mouse Fcgr1 gene is knocked out in a gene sequence. A gene sequence may be any sequence that is translated into messenger RNA. In some embodiments, a gene sequence is an exon, an intron, a combination of exons, a combination of introns, or a combination of exons and introns.

Mouse Fcgr1 has 6 exons, denoted exon 1-exon 6. In some embodiments, an immunodeficient mouse model provided herein has a deletion or modification in an exon of an endogenous Fcgr1 gene. For example, the mouse may have a deletion or modification in any one or more of exon 1, exon 2, exon 3, exon 4, exon 5, exon 6 of an endogenous Fcgr1 gene. In some embodiments, an immunodeficient mouse model has a deletion or modification in exons 3-6 of an endogenous Fcgr1 gene. In some embodiments, an immunodeficient mouse model has a deletion of exons 3-6 of an endogenous Fcgr1 gene.

In some embodiments, an immunodeficient Fcgr1^{null} mouse further comprises a human interleukin (IL-15) transgene. A transgene is a nucleic acid whose sequence is derived from a different species than the species that contains it (e.g., a human transgene in a mouse). A transgene may be expressed from a nucleic acid in a cell (e.g., in a mouse) or integrated into the genome of the mouse. Integration of a transgene (e.g., encoding human IL-15) may be by any genome editing method described herein. A transgene may be integrated into the genome of a mouse at the site of the mouse's orthologous gene (e.g., human IL-15 integrated into mouse IL-15 gene) or into another site in the genome of a mouse that does not disrupt mouse function (e.g., a safe-harbor locus such as Rosa26).

In some embodiments, an immuodeficient Fcgr1^{null} mouse transgenically expresses human interleukin-15 (Tg(hu-IL15)). Human interleukin 15 (IL-15) is a cytokine that plays a major role in the development of inflammatory and protective immune responses to microbial invaders and parasites by modulating immune cells of both the innate and adaptive immune systems. IL-15 plays a crucial role in the development, differentiation, and survival of natural killer (NK) cells that defend against viral infections, bacterial infections, and tumor cells. NK cells require IL-15 for their in vivo development and maintenance. NK cells are critical in antibody-dependent cellular cytotoxicity (ADCC). ADCC is an immune mechanism through which Fc receptor-bearing effector cells bind and kill antibody-coated target cells expressing tumor- or pathogen-derived antigens on their surface. An immunodeficient Fcgr1^{null} mouse transgenically expressing human interleukin-15 (Tg(hu-IL15)) will therefore have increased NK cell development and maintenance and ADCC activity compared with an immunodeficient Fcgr1^{null} mouse that does not express IL-15.

### Mouse Models

Herein, for simplicity, reference is made to “mouse” and “mouse models” (e.g., surrogates for human conditions). It should be understood that these terms, unless otherwise stated, may be used interchangeably throughout the specification to encompass “rodent” and “rodent models,” including mouse, rat, and other rodent species.

It should also be understood that standard genetic nomenclature used herein provides unique identification for different rodent strains, and the strain symbol conveys basic information about the type of strain or stock used and the genetic content of that strain. Rules for symbolizing strains and stocks have been promulgated by the International Committee on Standardized Genetic Nomenclature for Mice. The rules are available on-line from the Mouse Genome Database (MGD; informatics.jax.org) and were published in print copy (Lyon et al. 1996). Strain symbols typically include a Laboratory Registration Code (Lab Code). The first Lab Code appended to a strain symbol identifies and credits the creator of the strain. The Lab Code at the end of a strain symbol indicates the current source for obtaining mice of that strain. Different Lab Codes appended to the same strain symbol distinguish sublines and alert the user that there may be genetic divergence between the different sublines. Lab Codes are assigned from a central registry to assure that each is unique. The registry is maintained at the Institute for Laboratory Animal Research (ILAR) at the National Academy of Sciences, Washington, D.C. Lab Codes may be obtained electronically at ILAR's web site (nas.edu/cls/ilarhome.nsf). See also Davisson M T, Genetic and Phenotypic Definition of Laboratory Mice and Rats/What Constitutes an Acceptable Genetic-Phenotypic Definition, National Research Council (US) International Committee of the Institute for Laboratory Animal Research. Washington (DC): National Academies Press (US); 1999.

A mouse model of disease may be modified to enable the assessment of a disease. Any system (e.g., immune, respiratory, nervous, or circulatory), organ (e.g., blood, heart, blood vessels, spleen, thymus, lymph nodes, or lungs), tissue (e.g., epithelial, connective, muscle, and nervous), or cell type (e.g., lymphocytes or macrophages) may be modified, either independently or in combination, to enable studying disease in the models provided herein.

Three conventional methods used for the production of genome-modified mice (e.g., knockout mice, transgenic mice) include DNA microinjection (Gordon and Ruddle, Science 1981:214:1244-124, incorporated herein by reference), embryonic stem cell-mediated gene transfer (Gossler et al., Proc. Natl. Acad. Sci. 1986, 83:9065-9069, incorporated herein by reference) and retrovirus-mediated gene transfer (Jaenisch, Proc. Natl. Acad. Sci. 1976, 73:1260-1264, incorporated herein by reference), any of which may be used as provided herein. Genomic editing methods using, for example, clustered regularly interspace palindromic repeats (CRISPR/Cas) nucleases, transcription activator-like effector nucleases (TALENs), or zinc finger nucleases (ZFNs) are described elsewhere herein.

Following delivery of nucleic acids to a fertilized embryo (e.g., a single-cell embryo (e.g., a zygote) or a multi-cell embryo (e.g., a developmental stage following a zygote, such as a blastocyst), the fertilized embryo is transferred to a pseudopregnant female, which subsequently gives birth to offspring. The presence or absence of a nucleic acid encoding human FcRn and/or a chimeric IgG antibody may be confirmed, for example, using any number of genotyping methods (e.g., sequencing and/or genomic PCR).

New mouse models can also be created by breeding parental lines, as described in the Examples herein. With the variety of available mutant, knockout, knockin, transgenic, Cre-lox, Tet-inducible system, and other mouse strains, multiple mutations and transgenes may be combined to generate new mouse models. Multiple mouse strains may be bred together to generate double, triple, or even quadruple and higher multiple mutant/transgenic mice.

In some embodiments, parental mice are bred to produce F1 mice. A parental mouse may be, for example, homozygous, heterozygous, hemizygous, or homozygous null at a particular allele. Homozygous describes a genotype of two identical alleles at a given locus, heterozygous describes a genotype of two different alleles at a locus, hemizygous describes a genotype consisting of only a single copy of a particular gene in an otherwise diploid organism, and homozygous null refers to an otherwise-diploid organism in which both copies of the gene are missing.

### Immunodeficient Mouse Models

Provided herein, in some embodiments, are immunodeficient mouse models comprising a Fc gamma receptor 1 (null) Fcgr1^{null} allele and a mouse interleukin-2 receptor gamma null (IL-2Rγ^{null}) allele. An allele is a copy of a gene (e.g., Fcgr1) that contains a variation (e.g., Fcgr1^{null}) compared to a wild-type gene. In some embodiments, an immunodeficient mouse model comprises a Fcgr1^{null} allele, an IL-2Rγ^{null} allele, and a transgene encoding human IL-15. In some embodiments, an immunodeficient mouse is homozygous for the IL-2Rγ^{null} allele. In some embodiments, an immunodeficient mouse is homozygous for the Fcgr1^{null} allele. In some embodiments, an immunodeficient mouse is homozygous for the IL-2Rγ^{null} allele and is homozygous for the Fcgr1^{null} allele. Homozygous means that an immunodeficient mouse possesses two copies of an allele (e.g., Fcgr1^{null} allele, an IL-2Rγ^{null} allele). In some embodiments, an immunodeficient mouse comprises an interleukin-2 receptor gamma null (IL-2Rγ^{null}) allele. An IL-2Rγ^{null} allele is a null mutation in the gene encoding the interleukin 2 receptor gamma chain (IL2Rγ, homologous to IL2RG in humans), which blocks natural killer (NK) cell differentiation, thereby removing an obstacle that prevents the efficient engraftment of primary human cells (Cao et al., 1995; Greiner et al., 1998; and Shultz et al., 2005, each of which is incorporated herein by reference).

In some embodiments, an immunodeficient mouse comprises a Fcgr1^{null} allele and an IL-2Rγ^{null} allele further comprises a Prkdc^{scid} allele. The Prkdc^{scid} mutation is a loss-of-function (null) mutation in the mouse homolog of the human PRKDC gene—this mutation essentially eliminates adaptive immunity (see, e.g., (Blunt et al., 1995; Greiner, Hesselton, & Shultz, 1998), each of which is incorporated herein by reference). In some embodiments, an immunodeficient Fcgr1^{null}-IL-2Rγ^{null} mouse is homozygous for a Prkdc^{scid} allele.

In some embodiments, an immunodeficient mouse comprising a Fcgr1^{null} allele and an IL-2Rγ^{null} allele further comprises a Rag1^{null} allele. The Rag1^{null} mutation renders the mice B and T cell deficient. In some embodiments, an immunodeficient Fcgr1^{null}-IL-2Rγ^{null} mouse is homozygous for a Rag1^{null} allele.

As is known in the art, immunodeficient mice have impaired or disrupted immune systems, such as specific deficiencies in MHC class I, II or both, B cell or T cell defects, or defects in both, natural killer (NK) cell defects, myeloid defects (e.g., defects in granulocytes and/or monocytes), macrophage defects, dendritic cell defects, as well as immunodeficiency due to knockdown of genes for cytokines, cytokine receptors, TLR receptors and a variety of transducers and transcription factors of signaling pathways. Immunodeficiency mouse models include the single-gene mutation models such as nude-mice (nu) strains and the severe combined immunodeficiency (scid) strains, non-obese diabetic (NOD) strain, RAG (recombination activating gene) strains with targeted gene deletion and a variety of hybrids originated by crossing doubly and triple mutation mice strains with additional defects in innate and adaptive immunity.

An impaired immune system may be measured by any method known in the art including, but not limited to: production of mature immune cells (e.g., B cells, T cells, dendritic cells, macrophages, natural killer cells), deficient endogenous cytokine signaling, limited resistance to infection, and reduced survival. In some embodiments, an immunodeficient mouse lacks mature mouse T cells, lacks mature mouse B cells, lacks functional natural killer cells, and is deficient in endogenous (e.g., mouse) cytokine signaling. Mature T cells develop in the thymus and are released to other tissues, including blood, spleen, and lymphatic system. Mature B cells express pathogen-specific antibodies on their surface. Functional natural killer cells recognize and kill malignant and virally transformed cells without previously being exposed. Endogenous (e.g., mouse) cytokine signaling is important in maintaining homeostasis and relies on cytokines to regulate immune, nervous, and endocrine system function. Deficient endogenous (e.g., mouse) cytokine signaling means that the level of cytokine signaling is not sufficient to maintain immune system homeostasis compared to an endogenous immune system that is not deficient. Lack of mature cells (e.g., T cells or B cells), functional cells, (e.g., natural killer cells), deficient cytokine signaling, or some combination thereof may be a 10-99%, 5%-95%, 20%-90%, 30%-80%, 40%-70%, or 50%-60% decrease compared to a non-immunodeficient mouse. Lack of mature cells (e.g., T cells or B cells), functional cells (e.g., natural killer cells), deficient cytokine signaling, or some combination thereof may be a 10%, 15%, 20%, 25%, 30%, 35%, 40%, 45%, 50%, 55%, 60%, 65%, 70%, 75%, 80%, 85%, 90%, 95%, or 99% decrease compared to a non-immunodeficient mouse.

Lack of mature cells or functional cells (e.g., T cells, B cells, NK cells) may be assessed by any method known in the art including, but not limited to: flow cytometry; quantitative PCR (qPCR) of T cell markers (e.g., CD3, CD8, CD4, CD25, CD127, CD152), B cells markers (e.g., CD19, IgM, BCAP), and NK cells (e.g., CD224, CD122, NK11, NKp46, Ly49, CD11b, CD49b); immunofluorescence, and ELISA. Deficient cytokine signaling (e.g., mouse cytokine signaling) may be assessed by any method known in the art including, but not limited to: flow cytometry, qPCR of cytokines (e.g., IL-2, IL-7, IL-15, IFNγ, IL-4, IL-5, IL-9, IL-13, IL-25, IL-17A, IL-17F, IL-22, TNFα, IL-12, CCL3, GM-CSF, IL-6, IL-10, TGFβ, IL18, IL-21), immunofluorescence, and ELISA.

An immunodeficient mouse may express any human cytokine or combination of human cytokines that increases the efficacy of the immunodeficient mouse as an animal model (e.g., of human IgG antibody pharmacokinetics or activity, of human IgG antibody production, of human disease). A cytokine is a protein or peptide that modulates the activities of individual cells or tissues (e.g., other human cells, mouse cells). Non-limiting examples of types of human cytokines that may be expressed in a human fibrosis model include: hematopoietic cytokines, lymphokines, monokines, interferons, and chemokines.

In some embodiments, an immunodeficient mouse expresses a human hematopoietic cytokine. Human hematopoietic cytokines are extracellular proteins and peptides that stimulate hematopoietic cells (e.g., hematopoietic stem cells) to develop into differentiated blood cells (e.g., neutrophils, basophils, eosinophils, macrophage). Non-limiting examples of human hematopoietic cytokines include: interleukin 3 (IL-3), granulocyte/macrophage colony stimulating factor (GM-CSF), macrophage colony stimulating factor (M-CSF), thrombopoietin (TPO), IL-11, erythropoietin (EPO), granulocyte colony stimulating factor (G-CSF), IL-5, IL-6, IL-2, IL-7, IL-4, IL-17, and IL-15.

In some embodiments, an immunodeficient mouse described herein expresses 1-20, 2-19, 3-18, 4-17, 5-16, 6-15, 7-14, 8-13, 9-12, or 10-11 human cytokines (e.g., human hematopoietic cytokines). In some embodiments, an immunodeficient mouse described herein expresses 1, 2, 3, 4, 5, 6, 7, 8, 9, 10, 11, 12, 13, 14, 15, 16, 17, 18, 19, 20, or more human cytokines. In some embodiments, a human cytokine expressed in an immunodeficient mouse is involved in human antibody persistence and development.

A human cytokine may be expressed in an immunodeficient mouse by any method known in the art including, but not limited to: from a transgene in immunodeficient mouse and from a virus (e.g., lentivirus, adenovirus, adeno-associated virus) comprising a sequence encoding a human cytokine. A transgene is a gene that is transferred from one organism (e.g., human) to another (e.g., mouse). In some embodiments, a human cytokine is expressed from a transgene. A transgene in an immunodeficient mouse may be inserted into the immunodeficient mouse's genome or encoded in a vector that expresses the transgene.

Non-limiting examples of spontaneous and transgenic immunodeficient mouse models include the following mouse strains:
Nude (nu) [Flanagan S P. Genet Res 1966; 8:295-309; and Nehls M et al. Nature 1994; 372:103-7];
Scid (scid) [Bosma G C et al. Nature 1983; 301:527-30; Mosier D E et al. Nature 1988; 335:256-9; and Greiner D L et al. Stem Cells 1998; 16:166-77];
NOD [Kikutani H et al. Adv Immunol 1992; 51:285-322; and Anderson M S et al. Ann Rev Immunol 2005; 23:447-85];
RAG1 and RAG2 (rag) [Mombaerts P et al. Cell 1992; 68:869-77; Shinkai U et al. Cell 1992; 68:855-67];
NOD-scid [Greiner D L et al. 1998; Shultz L D et al. J Immunol 1995; 154:180-91; Melkus M W et al. Nature Med 2006; 12:1316-22; and Denton P W et al. PLOS Med 2008; 4 (12): e357];
IL2rgnull [DiSanto J P et al. Proc Natl Acad Sci USA 1995; 92:377-81];
B2mnull [Christianson S W et al. J Immunol 1997; 158:3578-86];
NOD-scid IL2rγnull [Shultz L D et al. Nat Rev Immunol 2007; 7:118-30; Ito M et al. Blood 2002; 100:3175-82; Ishikawa I et al. Blood 2005; 106:1565-73; and Macchiarini F et al. J Exp Med 2005; 202:1307-11];
NOD-scid B2mnull [Shultz et al. 2007; Shultz L D et al. Transplantation 2003; 76:1036-42; Islas-Ohlmayer M A et al. J Virol 2004; 78:13891-900; and Macchiarini et al. 2005];
HLA transgenic mice [Grusby M J et al. Proc Natl Acad Sci USA 1993; 90 (9): 3913-7; and Roy C J et al. Infect Immun 2005; 73 (4): 2452-60]. See, e.g., Belizario J E The Open Immunology Journal, 2009; 2:79-85;
NOG mice (NOD.cg-Prkdc^{scid} Il2rg^{tm1Sug}) [Shultz L D et al. Nat Rev Immunol 2007; 7:118-30]; and
BRG mice (BALB/c; 129S4-Rag2^{tm1.1Flv}) [Shultz L D et al. Nat Rev Immunol 2007; 7:118-30].

Provided herein, in some embodiments, are immunodeficient mouse models having the non-obese diabetic (NOD) mouse genotype. The NOD mouse (e.g., Jackson Labs Stock #001976, NOD-Shi^{LtJ}) is a polygenic mouse model of autoimmune (e.g., Type 1) diabetes, characterized by hyperglycemia and insulitis, a leukocytic infiltration of the pancreatic islet cells. The NOD mice are hypoinsulinemic and hyperglucagonemic, indicating a selective destruction of pancreatic islet beta cells. The major component of diabetes susceptibility in NOD mice is the unique MHC haplotype. NOD mice also exhibit multiple aberrant immunophenotypes including defective antigen presenting cell immunoregulatory functions, defects in the regulation of the T lymphocyte repertoire, defective NK cell function, defective cytokine production from macrophages (Fan et al., 2004) and impaired wound healing. They also lack hemolytic complement, C5. NOD mice also are severely hard-of-hearing. A variety of mutations causing immunodeficiencies, targeted mutations in cytokine genes, as well as transgenes affecting immune functions, have been backcrossed into the NOD inbred strain background.

In some aspects of the present disclosure, an immunodeficient mouse provided herein based on the NOD background has a genetic background (“background”) selected from NOD-Cg.-Prkdc^{scid} IL2rg^{tm1wJ1}/SzJ (NSG™), a NOD.Cg-Rag1^{tm1Mom} Il2rg^{tm1Wj1}/SzJ (NRG), and NOD.Cg-Prkdc^{scid}Il2rg^{tm1Sug}/ShiJic (NOG). Other immunodeficient mouse strains are contemplated herein.

In some embodiments, an immunodeficient mouse model based on the NOD background has an NOD-Cg.-Prkdc^{scid} IL2rg^{tm1wJ1}/SzJ (NSG™) genetic background. The NSG™ mouse (e.g., Jackson Labs Stock No.: #005557) is an immunodeficient mouse that lacks mature T cells, B cells, and NK cells, is deficient in multiple cytokine signaling pathways, and has many defects in innate immune immunity (see, e.g., Shultz, Ishikawa, & Greiner, 2007; Shultz et al., 2005; and Shultz et al., 1995, each of which is incorporated herein by reference). The NSG™ mouse, derived from the NOD mouse strain NOD/ShiLtJ (see, e.g., Makino et al., 1980, which is incorporated herein by reference), includes the Prkdc^{scid} mutation (also referred to as the “severe combined immunodeficiency” mutation or the “scid” mutation) and the Il2rg^{tm1Wj1} targeted mutation. The IL2rg^{tm1Wj1} mutation is a null mutation in the gene encoding the interleukin 2 receptor gamma chain (IL2Rγ, homologous to IL2RG in humans), which blocks NK cell differentiation, thereby removing an obstacle that prevents the efficient engraftment of primary human cells (Cao et al., 1995; Greiner et al., 1998; and Shultz et al., 2005, each of which is incorporated herein by reference).

In some embodiments, an immunodeficient mouse model has an NRG genotype. The NRG mouse (e.g., Jackson Labs Stock #007799) is extremely immunodeficient. This mouse comprises two mutations on the NOD/ShiLtJ genetic background; a targeted knockout mutation in recombination activating gene 1 (Rag1) and a complete null allele of the IL2 receptor common gamma chain (IL2rg^{null}). The extreme immunodeficiency of NRG allows the mice to be humanized by engraftment of human CD34⁺ hematopoietic stem cells (HSC) and patient derived xenografts (PDXs) at high efficiency. The immunodeficient NRG mice are more resistant to irradiation and genotoxic drugs than mice with a scid mutation in the DNA repair enzyme Prkdc.

In some embodiments, an immunodeficient mouse model is an NOG mouse. The NOG mouse (Ito M et al., Blood 2002) is an extremely severe combined immunodeficient (scid) mouse established by combining the NOD/scid mouse and the IL-2 receptor-γ chain knockout (IL2rγKO) mouse (Ohbo K. et al., Blood 1996). The NOG mouse lacks T and B cells, lacks natural killer (NK) cells, exhibits reduced dendritic cell function and reduced macrophage function, and lacks complement activity.

In some embodiments, an immunodeficient mouse model has an NCG genotype. The NCG mouse (e.g., Charles River Stock #572) was created by sequential CRISPR/Cas9 editing of the Prkdc and IL2rg loci in the NOD/Nju mouse, generating a mouse coisogenic to the NOD/Nju. The NOD/Nju carries a mutation in the Sirpa (SIRPα) gene that allows for engrafting of foreign hematopoietic stem cells. The Prkdc knockout generates a SCID-like phenotype lacking proper T-cell and B-cell formation. The knockout of the Il2rg gene further exacerbates the SCID-like phenotype while additionally resulting in a decrease of NK cell production.

Provided herein, in some embodiments, are immunodeficient mouse models that are deficient in MHC Class I, MHC Class II, or MHC Class I and MHC Class II. A mouse that is deficient in MHC Class I and/or MHC Class II does not express the same level of MHC Class I proteins (e.g., α-microglobulin and β2-microglobulin (B2M)) and/or MHC Class II proteins (e.g., α chain and β chain) or does not have the same level of MHC Class I and/or MHC Class II protein activity as a non-immunodeficient (e.g., MHC Class I/II wild-type) mouse. In some embodiments, the expression or activity of MHC Class I and/or MHC Class II proteins is reduced (e.g., by at least 30%, at least 40%, at least 50%, at least 60%, at least 70%, at least 80%, at least 90% or more), relative to a non-immunodeficient mouse.

Immunodeficient mice that are deficient in MHC Class I, MHC Class II, and MHC Class I and MHC Class II are described in International Publication No. WO 2018/209344, the contents of which are incorporated by reference herein.

An NSG-SGM3 mouse is the NSG derivative mouse NOD.Cg-Prkdc^{scid} Il2rg^{tm1Wj1} Tg(CMV-IL3,CSF2,KITLG)1Eav/MloySzJ (Jackson Laboratory Stock No: 013062). The transgenic NSG-SGM3 mice express three human cytokines: human Interleukin-3 (IL-3), human Granulocyte/Macrophage-colony stimulating factor 2 (GM-CSF), and human Stem Cell Factor (SCF). NSG-SGM3 mice combine the features of the highly immunodeficient NSG mouse with expression of human cytokines IL-3, GM-CSF, and SCF that support stable engraftment of myeloid lineages and regulatory T cell populations.

In some embodiments, an NSG mouse transgenically expresses human IL15. An NSG-IL-15 mouse, NOD.Cg-Prkdc^{scid} Il2rg^{tm1Wj1} Tg(IL15)1Sz/SzJ (Jackson Laboratory Stock No: 030890), expresses human IL15 and is combined with the highly immunodeficient NOD scid gamma (NSG) mouse. Expression of human IL15, in some embodiments, enhances the development of human NK cells in immunodeficient mice engrafted with human stem cells.

Therefore, the transgenic mice described herein may be produced by breeding an immunodeficient mouse comprising a Fc gamma receptor 1 (null) Fcgr1^{null} allele and a mouse interleukin-2 receptor gamma null (IL-2Rγ^{null}) allele. In some embodiments, the transgenic mice described herein are produced by knocking-out a Fcgr1 gene in an NSG-IL-15 mouse.

In some embodiments, an immunodeficient mouse is an NSG™ mouse with a Fcgr1 knockout (NSG-Fcgr1^{null}). In some embodiments, an NSG-Fcgr1^{null} mouse transgenically expresses a gene encoding a human interleukin 15 protein (NSG-Fcgr1^{null} Tg(Hu-IL15). An NSG-Fcgr1^{null} Tg(Hu-IL15) may express human IL-15 from any place in the mouse genome. In some embodiments, an NSG-Fcgr1^{null} Tg(Hu-IL15) mouse is produced by knocking-out Fcgr1 in an NSG-Tg(Hu-IL15) mouse described in U.S. application Ser. No. 16/637,621, which is incorporated by reference herein.

### Humanized Mouse Models

Provided herein, in some embodiments, are humanized immunodeficient mouse models and methods of producing the models. Immunodeficient mice engrafted with functional human cells and/or tissues are referred to as “humanized mice.” As used herein, the terms “humanized mouse”, “humanized immune deficient mouse”, “humanized immunodeficient mouse”, and the plural versions thereof are used interchangeably to refer to an immunodeficient mouse humanized by engraftment with functional human cells and/or tissues. For example, mouse models may be engrafted with human hematopoietic stem cells (HSCs) (e.g., CD34+ HSCs) and/or human peripheral blood mononuclear cells (PMBCs). In some embodiments, mouse models are engrafted with human tissues such as islets, liver, skin, and/or solid or hematologic cancers. In other embodiments, mouse models may be genetically modified such that endogenous mouse genes are converted to human homologs (see, e.g., Pearson, et al., Curr Protoc Immunol., 2008, Chapter: Unit-15.21).

Humanized mice are generated by starting with an immunodeficient mouse (e.g., an immunodeficient mouse of 2, 3, 4, 5, 6, 7, 8, 9, 10, or more weeks of age) and, if necessary, depleting and/or suppressing any remaining murine immune cells (e.g., chemically or with radiation). That is, successful survival of the human immune system in the immunodeficient mice may require suppression of the mouse's immune system to prevent GVHD (graft-versus-host disease) rejections. After the immunodeficient mouse's immune system has been sufficiently suppressed, the mouse is engrafted with human cells (e.g., HSCs and/or PBMCs). As used herein, “engraft” refers to the process of the human cells migrating to, and incorporating into, an existing tissue of interest in vivo. With respect to a humanized immunodeficient mouse, the engrafted human cells repopulate the mouse with a functional human hematopoietic system and/or human immune system.

The engrafted human cells (e.g., HSCs or PMBCs) for humanization, in some embodiments, are human leukocyte-antigen (HLA)-matched to the human cells (e.g., human cancer cells) of the mouse models. HLA-matched refers to cells that express the same major histocompatibility complex (MHC) genes. Engrafting mice with HLA-matched human xenografts and human immune cells, for example, reduces or prevents immunogenicity of the human immune cells. In some embodiments, a humanized mouse provided in the present disclosure is engrafted with human PMBCs or human HSCs that are HLA-matched to a PDX or human cancer cell line.

The engrafted human cells (e.g., HSCs or PMBCs) for humanization, in some embodiments, are not HLA-matched to the human cells (e.g., human cancer cells) of the mouse models. That is, in some embodiments, a humanized mouse provided in the present disclosure is engrafted with human PMBCs or human HSCs that are not HLA-matched to a PDX or human cancer cell line.

### Myeloablation

As described above, in some embodiments, immunodeficient mice are treated to deplete and/or suppress any remaining murine immune cells (e.g., chemically and/or with radiation). In some embodiments, immunodeficient mice are treated only chemically or only with radiation. In other embodiments, immunodeficient mice are treated both chemically and with radiation.

In some embodiments, immunodeficient mice are administered a myeloablative agent, that is, a chemical agent that suppresses or depletes murine immune cells. Examples of myeloablative agents include busulfan, dimethyl mileran, melphalan, and thiotepa.

In some embodiments, immunodeficient mice are irradiated prior to engraftment with human cells, such as human HSCs and/or PMBCs. It is thought that irradiation of an immunodeficient mouse destroys mouse immune cells in peripheral blood, spleen, and bone marrow, which facilitates engraftment of human cells, such as human HSCs and/or PMBCs (e.g., by increasing human cell survival factors), as well as expansion of other immune cells. Irradiation also shortens the time it takes to accumulate the required number of human immune cells to “humanize” the mouse models.

For immunodeficient mice (e.g., NSG™ mice), this preparation is commonly accomplished through whole-body gamma irradiation. Irradiators may vary in size depending on their intended use. Animals are generally irradiated for short periods of time (less than 15 min). The amount of time spent inside the irradiator varies depending on the radioisotope decay charts, amount of irradiation needed, and source of ionizing energy (that is, X-rays versus gamma rays, for which a cesium or cobalt source is needed).

A myeloablative irradiation dose is usually 700 to 1300 cGy, though in some embodiments, lower doses such as 1-100 cGy (e.g., about 2, 5, or 10 cGy), or 300-700 cGy may be used.

As an example, the mouse may be irradiated with 100 cGy X-ray (or 75 cGy-125 cGy X-ray). In some embodiments, the dose is about 1, 2, 3, 4, 5, 10, 20, 100, 125, 150, 175, 200, 300, 400, 500, 600, 700, 800, 900, 1000, 1100, 1200, or 1300 cGy, or between any of the two recited doses herein, such as 100-300 cGy, 200-500 cGy, 600-1000 cGy, or 700-1300 cGy. In some embodiments, the immunodeficient mouse is irradiated about 15 minutes, 30 minutes, 45 minutes, 1 hour, or more before engraftment with human HSCs and/or PMBCs. In some embodiments, the immunodeficient mouse is engrafted with human HSCs and/or PMBCs 1, 2, 3, 4, 5, 6, 7, 8, 9, 10, 11, 12, 13, 14, 15, 16, 17, or 18 days after irradiation.

### Engraftment (Humanization)

As described above, in some embodiments, the irradiated immunodeficient mice are engrafted with HSCs and/or PBMCs, humanizing the mice. Engraftment refers to the process of the human cells migrating to, and incorporating into, an existing tissue of interest in vivo. The PBMCs may be engrafted after irradiation and before engraftment of human diseased cells (e.g., human cancer cells), after irradiation and concurrently with engraftment of human diseased cells, or after irradiation and after engraftment of human diseased cells.

### PBMCs

Peripheral blood mononuclear cells (PBMCs) are peripheral blood cells having a round nucleus. These mononuclear blood cells recirculate between tissues and blood and are a critical component in the immune system to fight infection and adapt to intruders. There are two main types of mononuclear cells, lymphocytes and monocytes. The lymphocyte population of PBMCs typically includes T cells, B cells and NK cells.

PBMCs may be isolated from whole blood samples, for example (e.g., Ficoll gradient). In some embodiments, PBMCs from a subject (e.g., a human subject) with a current or previous diagnosis of cancer or an autoimmune disease may be used.

### HSCs

Hematopoietic stem cells (HSCs) are the stem cells that give rise to other blood cells during a process referred to as hematopoiesis. Hematopoietic stem cells give rise to different types of blood cells, in lines called myeloid and lymphoid. Myeloid and lymphoid lineages both are involved in dendritic cell formation. Myeloid cells include monocytes, macrophages, neutrophils, basophils, eosinophils, erythrocytes, and megakaryocytes to platelets. Lymphoid cells include T cells, B cells, natural killer cells, and innate lymphoid cells.

Methods of engrafting immunodeficient mice with HSCs and/or PBMCs to yield a humanized mouse model include but are not limited to intraperitoneal or intravenous injection (Shultz et al., J Immunol, 2015, 174:6477-6489; Pearson et al., Curr Protoc Immunol. 2008; 15-21; Kim et al., AIDS Res Hum Retrovirus, 2016, 32 (2): 194-2020; Yaguchi et al., Cell & Mol Immunol, 2018, 15:953-962). In some embodiments, the mouse is engrafted with 1.0×10⁶-3.0×10⁷ HSCs and/or PBMCs. In some embodiments, the mouse is engrafted with less than 1×10⁷ HSCs and/or PBMCs. In some embodiments, the mouse is engrafted with less than 1×10⁶ to about 5×10⁶ HSCs and/or PBMCs. In some embodiments, the mouse is engrafted with about 2×10⁶ or 1×10⁶ HSCs and/or PBMCs. In some embodiments, the mouse is engrafted with 25,000-100,000 HSCs (e.g., 25,000, 30,000, 35,000, 40,000, 45,000, 50,000, 55,000, 60,000, 65,000, 70,000, 75,000, 80,000, 85,000, 90,000, 95,000, 100,000 or more HSCs).

For example, the mouse may be engrafted with 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.5×10⁶, 3.0×10⁶ or more HSCs and/or PBMCs. In some embodiments, the mouse is engrafted with 1.0-1.1×10⁶, 1.0-1.2×10⁶, 1.0-1.3×10⁶, 1.0-1.4×10⁶, 1.0-1.5×10⁶, 1.0-1.6×10⁶, 1.0-1.7×10⁶, 1.0-1.8×10⁶, 1.0-1.9×10⁶, 1.0-2.0×10⁶, 1.0-2.25×10⁶, 1.0-2.5×10⁶, 1.0-2.75×10⁶, 1.0-3.0×10⁶, 1.1-1.2×10⁶, 1.1-1.3×10⁶, 1.1-1.4×10⁶, 1.1-1.5×10⁶, 1.1-1.6×10⁶, 1.1-1.7×10⁶, 1.1-1.8×10⁶, 1.1-1.9×10⁶, 1.1-2.0×10⁶, 1.1-2.25×10⁶, 1.1-2.5×10⁶, 1.1-2.75×10⁶, 1.1-3.0×10⁶, 1.2-1.3×10⁶, 1.2-1.4×10⁶, 1.2-1.5×10⁶, 1.2-1.6×10⁶, 1.2-1.7×10⁶, 1.2-1.8×10⁶, 1.2-1.9×10⁶, 1.2-2.0×10⁶, 1.2-2.25×10⁶, 1.2-2.5×10⁶, 1.2-2.75×10⁶, 1.2-3.0×10⁶, 1.3-1.4×10⁶, 1.3-1.5×10⁶, 1.3-1.6×10⁶, 1.3-1.7×10⁶, 1.3-1.8×10⁶, 1.3-1.9×10⁶, 1.3-2.0×10⁶, 1.3-2.25×10⁶, 1.3-2.5×10⁶, 1.3-2.75×10⁶, 1.3-3.0×10⁶, 1.4-1.5×10⁶, 1.4-1.6×10⁶, 1.4-1.7×10⁶, 1.4-1.8×10⁶, 1.4-1.9×10⁶, 1.4-2.0×10⁶, 1.4-2.25×10⁶, 1.4-2.5×10⁶, 1.4-2.75×10⁶, 1.4-3.0×10⁶, 1.5-1.6×10⁶, 1.5-1.7×10⁶, 1.5-1.8×10⁶, 1.5-1.9×10⁶, 1.5-2.0×10⁶, 1.5-2.25×10⁶, 1.5-2.5×10⁶, 1.5-2.75×10⁶, 1.5-3.0×10⁶, 1.6-1.7×10⁶, 1.6-1.8×10⁶, 1.6-1.9×10⁶, 1.6-2.0×10⁶, 1.6-2.25×10⁶, 1.6-2.5×10⁶, 1.6-2.75×10⁶, 1.6-3.0×10⁶, 1.7-1.8×10⁶, 1.7-1.9×10⁶, 1.7-2.0×10⁶, 1.7-2.25×10⁶, 1.7-2.5×10⁶, 1.7-2.75×10⁶, 1.7-3.0×10⁶, 1.8-1.9×10⁶, 1.8-2.0×10⁶, 1.8-2.25×10⁶, 1.8-2.5×10⁶, 1.8-2.75×10⁶, 1.8-3.0×10⁶, 1.9-2.0×10⁶, 1.9-2.25×10⁶, 1.9-2.5×10⁶, 1.9-2.75×10⁶, 1.9-3.0×10⁶, 2.0-2.25×10⁶, 2.0-2.5×10⁶, 2.0-2.75×10⁶, 2.0-3.0×10⁶, 2.25-2.5×10⁶, 2.25-2.75×10⁶, 2.25-3.0×10⁶, 2.5-2.75×10⁶, 2.5-3.0×10⁶, or 2.75-3.0×10⁶ HSCs and/or PBMCs.

In some embodiments, the mouse is engrafted with a dose of less than 1×10⁷ PBMCs (e.g., 1×10⁶ to about 5×10⁶ PBMCs, about 2×10⁶ PBMCs, or about 1×10⁶ PBMCs).

As described herein, in some embodiments, engraftment with HSCs and/or PBMCs yields a transgenic mouse comprising more human CD45+ cells in peripheral blood, relative to a humanized NSG or NSG-Tg(Hu-IL15) control mouse (e.g., an increase of 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40% or more). In embodiments, the transgenic mouse comprises significantly more human NK cells in peripheral blood, relative to a humanized NSG or NSG-Tg(Hu-IL15) control mouse (e.g., an increase of 0.5%, 1%, 2%, 3%, 4%, 5%, 6%, 7%, 8%, 9%, 10%, or more, measured as a percentage of CD45+ cells).

In some embodiments, engraftment with PBMCs yields a transgenic mouse comprising more human myeloid cells in peripheral blood relative to a humanized NSG or NSG-Tg(Hu-IL15) control mouse (e.g., an increase of 0.01%, 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2% or more, measured as a percentage of CD45+ cells).

In some embodiments, the transgenic mouse comprises more activated human T cells relative to a humanized NSG or NSG-Tg(Hu-IL15) control mouse. In some embodiments, the activated T cells are present in the spleen of the transgenic mouse. The term “T-cell activation” refers to the mechanisms of activation of T-cells which may vary slightly between different types of T cells. The “two-signal model” in CD4+ T cells, however, is applicable for most types of T-cells. In more detail, activation of CD4+ T cells typically occurs through the engagement of both the T cell receptor and CD28 on the T cell surface by the major histocompatibility encoded antigen-presenting molecule and with its bound antigenic peptide and B7 family members on the surface of an antigen presenting cell (APC), respectively. Both cell-cell contacts are generally required for the production of an effective immune response. For example, in the absence of CD28 co-stimulation, T-cell receptor signaling alone may result in T-cell anergy. The further signaling pathways downstream from both CD28 and the T cell receptor involve many further proteins known to the skilled person. The activation of T-cells may be determined by cytokine release and/or cell proliferation, in particular, proliferation of T-cells.

### Methods of Use

### Human Antibody Pharmacokinetics

The present disclosure provides, in some aspects, methods of administering an antibody to an immunodeficient Fcgr1^{null} mouse and assaying the level of the antibody. An antibody may be a monoclonal antibody or a polyclonal antibody. A monoclonal antibody is an antibody produced by a single clone of cells or cell line and consists of identical antibody molecules. A polyclonal antibody is an antibody produced by different B cell lineages and includes of a collection of immunoglobulin molecules that react against a specific antigen.

In some embodiments, an antibody is a human or humanized antibody. A human antibody is an antibody is an antibody containing only human antibody sequences. A humanized antibody is an antibody containing sequences from non-human species. In some embodiments, a humanized antibody's sequence has been modified to more closely align with antibodies produced in humans.

In some embodiments, an antibody is an immune checkpoint inhibitor (ICI) antibody. An immune checkpoint is a regulator for the immune system, that prevents the immune system from attacking cells indiscriminately. The immune checkpoint prevents immune cells from attacking certain cells. Immune checkpoint inhibitors (ICIs) are molecules the inhibit the immune checkpoint and therefore allow the immune system to attack certain cells (e.g., cancer cells). An ICI may be any ICI known in the art. Non-limiting examples of immune checkpoint inhibitors include: programmed death 1 (PD-1), cytotoxic T-lymphocyte-associated protein 4 (CTLA4), adenosine A2A receptor (A2AR), B7-H3, B7-H4, B and T lymphocyte attenuator (BTLA), indoleamine 2,3-dioxygenase (IDO), killer-cell immunoglobulin like-receptor (KIR), lymphocyte activation gene-3 (LAG3), nicotinamide adenine dinucleotide phosphate oxidase isoform 2 (NOX2), T-cell immunoglobulin domain and mucin domain 3 (TIM3), V-domain Ig suppressor of T cell activation (VISTA), and sialic acid-binding immunoglobulin-type lectin 7 (SIGLEC7).

In some embodiments, an ICI antibody is an anti-PD-1 antibody. PD-1 (also known as CD279) is a protein on the surface of T and B cells that inhibits the immune system's response to cells of the human body. PD-1 inhibits the immune response by increasing apoptosis of antigen-specific T-cells in lymph nodes and by reducing apoptosis in regulatory T-cells. A human anti-PD-1 antibody may agonize (increase) or antagonize (decrease) the activity of PD-1. An antibody that agonizes the activity of PD-1 is useful to further inhibit the immune system's response to cells of the human body and may be useful in treating a disease in which the immune system is overactive (e.g., autoimmune disease). An antibody that antagonizes the activity of PD-1 is useful to allow the immune system to attack cells of the human body and may be useful in treating a disease in which the human body's cells are not undergoing apoptosis or are undergoing apoptosis at a decreased rate compared to a control (e.g., cancer). An anti-PD-1 antibody may be any anti-PD-1 antibody known in the art. Non-limiting examples of anti-PD-1 antibodies include: pembrolizumab (Keytruda®), nivolumab (Opdivo®), pidilizumab (CT-011), toripalimab (JS-001) cemiplimab (Libtayo®), dostarlimab (Jemperli®), camrelizumab (SHR1210), sintilimab (IBI308), tislelizumab (BGB-A317), and INCMGA00012 (MGA012).

In some embodiments, an ICI antibody is an anti-PD-L1 antibody. PD-L1 (also known as CD274 and B7-H1) is a transmembrane protein that binds to PD-1. Binding of PD-L1 to PD-1 produces an inhibitory signal that reduces the proliferation of antigen-specific T-cells in lymph nodes, while simultaneously reducing apoptosis in regulatory T-cells. An anti-PD-L1 antibody may agonize (increase) or antagonize (decrease) the activity of PD-L1. An antibody that agonizes the activity of PD-L1 is useful to further inhibit the immune system's response to cells of the human body and may be useful in treating a disease in which the immune system is overactive (e.g., autoimmune disease). An antibody that antagonizes the activity of PD-L1 is useful to allow the immune system to attack cells of the human body and may be useful in treating a disease in which the human body's cells are not undergoing apoptosis or are undergoing apoptosis at a decreased rate compared to a control (e.g., cancer). An anti-PD-L1 antibody may be any anti-PD-L1 antibody known in the art. Non-limiting examples of anti-PD-L1 antibodies include: atezolizumab (Tecentriq®), avelumab (Bavencio®), durvalumab (Imfinzi®), and KN035.

In some embodiments, an ICI is an anti-CTLA-4 antibody. CTLA-4 (also known as CD152) is constitutively expressed on regulatory T cells and downregulates immune responses. An anti-CTLA-4 antibody may agonize (increase) or antagonize (decrease) the activity of CTLA-4. An antibody that agonizes the activity of CTLA-4 is useful to reduce immune activity and may be useful in treating a disease in which the immune system is overactive (e.g., autoimmune disease). An antibody that antagonizes the activity of CTLA-4 is useful to allow the immune system to attack cells of the human body and may be useful in treating a disease in which the human body's cells are not undergoing apoptosis or are undergoing apoptosis at a decreased rate compared to a control (e.g., cancer). An anti-CTLA-4 antibody may be any anti-CTLA-4 antibody known in the art. Non-limiting examples of anti-CTLA-4 antibodies include: ipilimumab (Yervoy®), L3D10, and tremelimumab (CP-675,206).

In some embodiments, an antibody is a human immunoglobulin (Ig) antibody. A human Ig antibody may be an immunoglobulin gamma (IgG), an immunoglobulin alpha (IgA), an immunoglobulin beta (IgB), an immunoglobulin epsilon (IgE), or an immunoglobulin mu (IgM) antibody. In some embodiments, a human Ig antibody is an IgG antibody. IgG antibodies may be any IgG antibody described herein.

In some embodiments, an immunodeficient Fcgr1^{null} mouse is a non-obese diabetic (NOD) immunodeficient mouse. Human IgGs have a greatly shortened half-life in NOD mice compared to other immunodeficient mouse strains, including CB17-scid and BALB/c-nude (Li et al., Molecular Cancer Therapeutics, 2019; 18(4): 780-787). This shortened half-life in NOD mice is due to an abnormality in the NOD Fc gamma R1 (FcγRI) receptor (also known as CD64). The NOD strain background expresses the “d” allele at the Fcgr1 locus that encodes an abnormally highly active FcgR1 receptor with an unusually high affinity for human IgG (Gavin et al., Immunogenetics, 2000; 51 (3): 206-211). This increased affinity was found to be due to a gain of function mutations in the NOD mouse strain Fcgr1 gene (Gavin et al., EMBO Journal, 1998; 17 (14): 3850-3857).

Any mouse with similar gain of function mutations in an Fcgr1 gene (or an ortholog thereof) that has been knocked out would be similarly useful in methods provided herein to study human IgG antibody pharmacokinetics and activity because without an abnormally active FcgR1 receptor that has an unusual affinity for human IgG antibodies, the mouse would more faithfully recapitulate human IgG antibody recycling and activity.

Antibody pharmacokinetics may include any pharmacokinetic parameter assayed in the art. Non-limiting examples of pharmacokinetic parameters include: antibody distribution (e.g., in tissues, across blood vessels, across barriers, in cell types), half-life, elimination route (e.g., renal, hepatic), routes of administration (e.g., intravenous, subcutaneous, intramuscular), and bioavailability (e.g., metabolism in mouse, solubility in blood, ability to reach target cells).

In some embodiments, antibody half-life is assayed. Antibody half-life is the length of time for the concentration of an antibody (e.g., in a mouse) to decrease by half. As provided herein, the half-life of a human IgG antibody is longer in an immunodeficient Fcgr1^{null} mouse than in its wild-type or immunodeficient counterpart. Half-life may be studied by any method known in the art. Non-limiting methods of studying antibody half-life include: enzyme-linked immunosorbent assay (ELISA), western blot, and surface plasmon resonance (SPR).

In some embodiments, the half-life of an antibody is increased 2-fold-100-fold, 5-fold-95-fold, 10-fold-90-fold, 15-fold-85-fold, 20-fold-80-fold, 25-fold-75-fold, 30-fold-70-fold, 35-fold-65-fold, 40-fold-60-fold, or 45-fold-55-fold relative to a control. In some embodiments, the half-life of an antibody is increased 2-fold, 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold or more, relative to a control. A control may be, for example, a mouse (e.g., a wild-type or immunodeficient mouse) that expresses functional mouse FcγR1.

In some embodiments, the circulating level of an antibody is assayed in a biological sample obtained from a mouse administered the antibody. The circulating level of an antibody is the amount of the antibody present in the mouse's circulation at a given time period (e.g., after administration of the antibody). An antibody's circulating level may be compared to a control (e.g., a mouse that does not comprise an Fcgr1^{null} allele) to study the pharmacokinetics of the antibody over time.

A biological sample may be any biological sample obtained from a mouse. Non-limiting examples of a biological sample include: blood, serum, plasma, lymph, cerebrospinal fluid, urine, feces, and tissue. A biological sample may be obtained from a mouse by any method known in the art. Non-limiting methods of obtaining a biological sample include: intravenous withdrawal, intraarterial withdrawal, collecting urine, spinal tap, lymphatic system drainage, collecting feces, and biopsy.

The circulating level of an antibody (e.g., for assaying half-life) may be assayed multiple times over a given time period. In some embodiments, the level of an antibody is assayed 2-25 times, 3-24 times, 4-23 times, 5-22 times, 6-21 times, 7-20 times, 8-19 times, 9-18 times, 10-17 times, 11-16 times, 12-15 times, or 13-14 times. In some embodiments the level of an antibody is assayed 2, 3, 4, 5, 6, 7, 8, 9, 10, 11, 12, 13, 14, 15, 16, 17, 18, 19, 20, 21, 22, 23, 24, or 25 or more times. In some embodiments, the level of an antibody is assayed multiple times over 1 day-30 weeks, 3 days-29 weeks, 7 days-28 weeks, 1.5 weeks-27 weeks, 2 weeks-26 weeks, 2.5 weeks-25 weeks, 3 weeks-24 weeks, 3.5 weeks-23 weeks, 4 weeks-22 weeks, 4.5 weeks-21 weeks, 5 weeks-20 weeks, 6 weeks-19 weeks, 7 weeks-18 weeks, 8 weeks-17 weeks, 9 weeks-16 weeks, 10 weeks-15 weeks, 11 weeks-14 weeks, or 12-13 weeks. In some embodiments, the level of an antibody is assayed multiple times over 1 day, 3 days, 7 days, 1.5 weeks, 2 weeks, 2.5 weeks, 3 weeks, 3.5 weeks, 4 weeks, 4.5 weeks, 5 weeks, 5.5 weeks, 6 weeks, 6.5 weeks, 7 weeks, 7.5 weeks, 8 weeks, 8.5 weeks, 9 weeks, 9.5 weeks, 10 weeks, 10.5 weeks, 11 weeks, 11.5 weeks, 12 weeks, 12.5 weeks, 13 weeks, 13.5 weeks, 14 weeks, 14.5 weeks, 15 weeks, 15.5 weeks, 16 weeks, 16.5 weeks, 17 weeks, 17.5 weeks, 18 weeks, 18.5 weeks, 19 weeks, 19.5 weeks, 20 weeks, 20.5 weeks, 21 weeks, 21.5 weeks, 22 weeks, 22.5 weeks, 23 weeks, 23.5 weeks, 24 weeks, 24.5 weeks, 25 weeks, 25.5 weeks, 26 weeks, 26.5 weeks, 27 weeks, 27.5 weeks, 28 weeks, 28.5 weeks, 29 weeks, 29.5 weeks, or 30 weeks or longer.

In some embodiments, a human antibody is an IgG antibody. A human IgG antibody may be any human IgG antibody known in the art. Non-limiting examples of human IgG antibodies include immunoglobulin 1 (IgG1), immunoglobulin 2 (IgG2), immunoglobulin 3 (IgG3), and immunoglobulin 4 (IgG4). In some embodiments, a human IgG antibody is an IgG1 antibody, an IgG4 antibody, or a combination thereof.

More than one antibody (e.g., human IgG antibody) may be administered to an immunodeficient Fcgr1^{null} mouse. In some embodiments, 1-20 antibodies, 2-19 antibodies, 3-18 antibodies, 4-17 antibodies, 5-16 antibodies, 6-15 antibodies, 7-14 antibodies, 8-13 antibodies, 9-12 antibodies, or 10-11 antibodies are administered to an immunodeficient Fcgr1^{null} mouse. In some embodiments, 1, 2, 3, 4, 5, 6, 7, 8, 9, 10, 11, 12, 13, 14, 15, 16, 17, 18, 19, or 20 or more antibodies are administered to an immunodeficient Fcgr1^{null} mouse.

In some embodiments, an antibody is a therapeutic human IgG antibody. A therapeutic human IgG antibody is an antibody that is known to or thought to treat or prevent a human disorder. A therapeutic human IgG antibody may be any human IgG antibody known in the art (e.g., IgG1, IgG2, IgG3, IgG4). Non-limiting examples of therapeutic human IgG antibodies include: trastuzumab (Herceptin®), pembrolizumab (Keytruda®), adaluminab (Humira®), alemtuzumab (Lemtrada®), alirocumab (Praluent®), basiliximab (Simulect®), belimumab (Benlysta®), bevacizumab (Avastin®), canakinumab (Ilaris®), cetuximab (Erbitux®), daclizumab (Zinbryta®), daratumumab (Darzalex®), denosumab (Prolia®), dinutuximab (Unituxin®), eculizumab (Soliris®), elotuzumab (Empliciti®), evolocumab (Repatha®), golimumab (Simponi®), infliximab (Remicade®), ipilimumab (Yervoy®), ixekizumab (Taltz®), mepolizumab (Nucala®), natalizumab (Tysabri®), necitumumab (Portrazza®), nivolumab (Opdivo®), obiltoxaximab (Anthim®), Obinutuzumab (Gazyva®), ofatumumab (Arzerra®), olaratumab (Lartruvo®), omalizumab (Xolair®), palivizumab (Synagis®), panitumumab (Vectibix®), pertuzumab (Perjeta®), ramucirumab (Cyramza®), ranibizumab (Lucentis®), raxibacumab (Abthrax®), reslizumab (Cinqair®), rituximab (Rituxan®), secukinumab (Cosentyx®), siltuximab (Sylvant®), tocilizumab (Actemra®), altizumab (RoActemra®), tositumomab (Bexxar®), ustekinumab (Stelara®), vedolizumab (Entyvio®), L3D10, tremelimumab (CP-675,206), atezolizumab (Tecentriq®), avelumab (Bavencio®), durvalumab (Imfinzi®), KN035, pidilizumab (CT-011), toripalimab (JS-001) cemiplimab (Libtayo®), dostarlimab (Jemperli®), camrelizumab (SHR1210), sintilimab (IBI308), tislelizumab (BGB-A317), and INCMGA00012 (MGA012).

The route of administration of an antibody to an immunodeficient Fcgr1^{null} mouse is not limited. Non-limiting examples of routes of administration include: subcutaneous injection, intravenous injection, intramuscular injection, intraarterial injection, intraperitoneal injection, intracranial injection, intraventricular injection, inhalation, and ingestion.

### Antibody Production

One limitation of a mouse that has an overactive Fc gamma receptor (FcgR) (e.g., NSG mouse) is that these mice do not produce adequate human IgG antibodies after engraftment with human immune cells to model a human immune response. The present disclosure provides, in some embodiments, a method of engrafting an immunodeficient Fcgr1^{null} mouse with human hematopoietic stem cells (HSCs) and/or peripheral blood mononuclear cells (PMBCs) and measuring the level of a human IgG antibody in an immunodeficient Fcgr1^{null} mouse. The Fcgr1^{null} mouse may be any mouse described herein. In some embodiments, a mouse is engrafted with HSCs. In some embodiments, a mouse is engrafted with PMBCs. In some embodiments, a mouse is engrafted with HSCs and PMBCs.

HSCs and PMBCs are cells that give rise to other blood cells in a process known as hematopoiesis. Engrafting a mouse with human HSCs and/or PMBCs allows the non-human animal to produce human blood cells. When a mouse produces human blood cells, the mouse may also produce human antibodies (e.g., human IgGs). This is particularly true when the mouse is immunodeficient prior to being engrafted with human HSCs. Immunodeficiency is discussed in greater detail in the “Mouse Models” section below. Thus, engrafting an immunodeficient Fcgr1^{null} mouse with human HSCs and/or PMBCs enables the mouse to model human immune behavior.

An antigen may be administered to a non-human animal engrafted with human HSCs to provoke a human immune response. This antigen may be any antigen that will produce an immune response in humans. Non-limiting examples of such antigens include: human tumor cells, bacterial cells, viruses, and fungal cells.

Human HSCs and PMBCs are described in previously.

The half-life and circulating level of any human antibody in an immunodeficient Fcgr1^{null} mouse engrafted with human HSCs and/or PMBCs may be measured by any of the methods and with any frequency and duration provided herein.

In some embodiments, the circulating level of a human antibody is increased 2-fold-50-fold, 5-fold-45-fold, 10-fold-40-fold, 15-fold-35-fold, 20-fold-35-fold, or 25-fold-35-fold relative to a control. In some embodiments, the circulating level of a human IgG antibody is increased 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, or 50-fold or more relative to a control. A control may be a wild-type Fcgr1 mouse or an immunodeficient Fcgr1^{null} mouse that has not been engrafted with HSCs and/or PMBCs.

### Disease Treatment

Human antibodies are used to treat and prevent a wide variety of diseases, including cancer, rheumatoid arthritis (RA), Crohn's disease (CD), multiple sclerosis (MS), heterozygous and homozygous familial hypercholesteremia (HeFH, HoFH), transplant rejection, systemic lupus erythematosus (SLE), paroxysmal nocturnal hemoglobinuria, asthma, macular degeneration, plaque psoriasis (PP), psoriatic arthritis (PA), and ulcerative colitis (UC). Identifying new human therapeutic antibodies and new diseases that are treated or prevented by human therapeutic antibodies is a critical area of research. The present disclosure provides, in some embodiments, administering to an immunodeficient Fcgr1^{null} mouse having a human disease a therapeutic antibody and/or putative therapeutic antibody and measuring disease proliferation or survival in the Fcgr1^{null} mouse.

In some embodiments, an immunodeficient Fcgr1^{null} mouse is engrafted with human diseased cells. Engrafting may be by any method provided herein. Human diseased cells are cells derived from a human (e.g., patient-derived xenograft or human cell line) and associated with a disease. Associated with a disease may be confirmed by any method known in the art. Non-limiting examples of confirming whether a human cell is associated with a disease are: detecting or measuring whether the cell(s) expresses a mutant gene or protein (e.g., BRCA1, p53, etc) associated with the disease, detecting increased proliferation in the human cell compared to a control, detecting decreased apoptosis in the human cell compared to a control, and detecting increased mobility in the human cell compared to a control. A control may be a human cell derived from the same human or same cell population that is known to not be associated with a disease (e.g., a wild-type human cell).

Human diseased cells may be derived from any human disease known in the art. Non-limiting examples of human disease include: cancers (e.g., breast, lung, colorectal, melanoma), autoimmune disorders (e.g., RA, CD, MS, SLE, UC, PP, PA), genetic disorders (e.g., HeFH, HoFH, cystic fibrosis, Down syndrome, Duchenne muscular dystrophy), and infectious diseases (e.g., coronavirus disease 19 (COVID-19), hepatitis B virus, rabies virus, respiratory syncytial virus, Clostridium tetani, Clostridium botulinum, vaccinia virus, echovirus, enterovirus).

In some embodiments, a human disease is cancer and the human diseased cells are human tumor cells. Human tumor cells are cells that make up or are associated with a tumor. A tumor is an abnormal growth or accumulation of cells and may be either malignant or benign. A human cancer may be any cancer known in the art. Non-limiting examples of human cancers include: adenoid cystic carcinoma, adrenal gland tumor, amyloidosis, anal cancer, appendix cancer, astrocytoma, ataxia-telegiectasia, Beckwith-Wiedeman syndrome, bile duct cancer, Birt-Hogg-Dube syndrome, bladder cancer, bone cancer, brain stem glioma, brain cancer, breast cancer, Carney complex, cervical cancer, colorectal cancer, Cowden Syndrome, craniopharyngioma, desmoid tumor, desmoplastic infantile ganglioglioma, ependymoma, esophageal cancer, Ewing sarcoma, eye cancer, eyelid cancer, familial adenomatous polyposis, familial GIST, familial malignant melanoma, familial pancreatic cancer, gallbladder cancer, gastrointestinal stromal tumor (GIST), germ cell tumor, gestational trophoblastic disease, head and neck cancer, hereditary breast and ovarian cancer, hereditary diffuse gastric cancer, hereditary leiomyomatosis and renal cell cancer, hereditary mixed polyposis syndrome, hereditary pancreatitis, hereditary papillary renal carcinoma, HIV/AIDS-related cancer, juvenile polyposis syndrome, kidney cancer, lacrimal gland tumor, laryngeal and hypopharyngeal cancer, leukemia, Li-Fraumeni syndrome, liver cancer, lung cancer, lymphoma, Lynch syndrome, mastocytosis, medullablastoma, melanoma, meningioma, mesothelioma, multiple endocrine neoplasia type 1, multiple endocrine neoplasia type 2, multiple myeloma, MUTYH-associated polyposis, myelodysplastic syndrome, nasal cavity and paranasal sinus cancer, nasopharyngeal cancer, neuroblastoma, neuroendocrine tumor of the gastrointestinal tract, neuroendocrine tumor of the lung, neuroendocrine tumor of the pancreas, neurofibromatosis type 1, neurofibromatosis type 2, nevoid basal cell carcinoma syndrome, oral and oropharyngeal cancer, osteosarcoma, ovarian cancer, pancreatic cancer, parathyroid cancer, penile cancer, Peutz-Jeghers syndrome, pheochromocytoma and paraganglioma, pituitary gland tumor, pleuropulmonary blastoma, prostate cancer, retinoblastoma, rhabdomyosarcoma, salivary gland cancer, sarcoma, non-melanoma skin cancer, small bowel cancer, stomach cancer, testicular cancer, thymoma and thymic carcinoma, thyroid cancer, tuberous sclerosis complex, uterine cancer, vaginal cancer, Von Hippel-Lindau syndrome, vulvar cancer, Waldenstrom Macroglobulinemia, Werner syndrome, Wilms tumor, and xeroderma pigmentosum.

In some embodiments, a human cancer is produced in a mouse by engrafting an immunodeficient Fcgr1^{null} mouse with human cancer cells. Human cancer cells may be engrafted by any method provided herein. Human cancer cells may be patient-derived xenografts (PDXs) or cultured human cancer cells.

A human IgG antibody may be any human IgG antibody provided herein. In some embodiments, a human IgG antibody is an IgG1 antibody, an IgG2 antibody, an IgG3 antibody, an IgG4 antibody, or a combination thereof.

Disease proliferation may be any aspect of the disease pathology. Non-limiting examples of disease proliferation include: growth of human diseased cells, survival of human diseased cells, mobility of human diseased cells, and divisional of human diseased cells relative to a control. A control may be A control may be a human cell derived from the same human or same cell population that is known to not be associated with a disease (e.g., a wild-type human cell).

Disease proliferation and/or survival may be measured by any method known in the art. Methods of measuring disease proliferation will depend on the disease being studied. Non-limiting methods of measuring disease proliferation include: immunostaining, immunohistochemistry, enzyme-linked immunosorbent assay (ELISA), and imaging (e.g., magnetic resonance imaging, positron emission tomography, computerized tomography, X-ray imaging).

In some embodiments, disease proliferation and/or survival in an immunodeficient Fcgr1^{null} mouse is decreased 1%-200%, 5%-195%, 10%-190%, 15%-185%, 20%-180%, 25%-175%, 30%-170%, 35%-165%, 40%-160%, 45%-155%, 50%-150%, 55%-145%, 60%-140%, 65%-135%, 70%-130%, 75%-125%, 80%-120%, 85%-115%, 90%-110%, or 95%-105% compared to a control. In some embodiments, disease proliferation and/or survival in an immunodeficient Fcgr1^{null} mouse is decreased 1%, 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% compared a control. A control may be a wild-type Fcgr1 mouse, an immunodeficient Fcgr1^{null} mouse not injected with a human IgG antibody, or the Fcgr1^{null} mouse before being treated with the human IgG antibody.

Disease proliferation and/or survival may be measured multiple times over a given time period. In some embodiments, disease proliferation and/or survival is measured 2-25 times, 3-24 times, 4-23 times, 5-22 times, 6-21 times, 7-20 times, 8-19 times, 9-18 times, 10-17 times, 11-16 times, 12-15 times, or 13-14 times. In some embodiments disease proliferation and/or survival is measured 2, 3, 4, 5, 6, 7, 8, 9, 10, 11, 12, 13, 14, 15, 16, 17, 18, 19, 20, 21, 22, 23, 24, or 25 or more times. In some embodiments, disease proliferation and/or survival is measured multiple times over 1 day-30 weeks, 3 days-29 weeks, 7 days-28 weeks, 1.5 weeks-27 weeks, 2 weeks-26 weeks, 2.5 weeks-25 weeks, 3 weeks-24 weeks, 3.5 weeks-23 weeks, 4 weeks-22 weeks, 4.5 weeks-21 weeks, 5 weeks-20 weeks, 6 weeks-19 weeks, 7 weeks-18 weeks, 8 weeks-17 weeks, 9 weeks-16 weeks, 10 weeks-15 weeks, 11 weeks-14 weeks, or 12-13 weeks. In some embodiments, the disease proliferation and/or survival is measured multiple times over 1 day, 3 days, 7 days, 1.5 weeks, 2 weeks, 2.5 weeks, 3 weeks, 3.5 weeks, 4 weeks, 4.5 weeks, 5 weeks, 5.5 weeks, 6 weeks, 6.5 weeks, 7 weeks, 7.5 weeks, 8 weeks, 8.5 weeks, 9 weeks, 9.5 weeks, 10 weeks, 10.5 weeks, 11 weeks, 11.5 weeks, 12 weeks, 12.5 weeks, 13 weeks, 13.5 weeks, 14 weeks, 14.5 weeks, 15 weeks, 15.5 weeks, 16 weeks, 16.5 weeks, 17 weeks, 17.5 weeks, 18 weeks, 18.5 weeks, 19 weeks, 19.5 weeks, 20 weeks, 20.5 weeks, 21 weeks, 21.5 weeks, 22 weeks, 22.5 weeks, 23 weeks, 23.5 weeks, 24 weeks, 24.5 weeks, 25 weeks, 25.5 weeks, 26 weeks, 26.5 weeks, 27 weeks, 27.5 weeks, 28 weeks, 28.5 weeks, 29 weeks, 29.5 weeks, or 30 weeks or longer.

### Nucleic Acids: Engineering and Delivery

A mouse described herein comprises a nucleic acid encoding human interleukin-15 (IL-15) and a nucleic acid comprising a Fcgr1^{null} allele. In some embodiments, the mouse comprises a transgene encoding human IL-15, integrated into a genome, and comprises a Fcgr1^{null} allele in the genome.

The nucleic acids provided herein, in some embodiments, are engineered. An engineered nucleic acid is a nucleic acid (e.g., at least two nucleotides covalently linked together, and in some instances, containing phosphodiester bonds, referred to as a phosphodiester backbone) that does not occur in nature. Engineered nucleic acids include recombinant nucleic acids and synthetic nucleic acids. A recombinant nucleic acid is a molecule that is constructed by joining nucleic acids (e.g., isolated nucleic acids, synthetic nucleic acids or a combination thereof) from two different organisms (e.g., human and mouse). A synthetic nucleic acid is a molecule that is amplified or chemically, or by other means, synthesized. A synthetic nucleic acid includes those that are chemically modified, or otherwise modified, but can base pair with (bind to) naturally occurring nucleic acid molecules. Recombinant and synthetic nucleic acids also include those molecules that result from the replication of either of the foregoing.

An engineered nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

In some embodiments, a nucleic acid is a complementary DNA (cDNA). cDNA is synthesized from a single-stranded RNA (e.g., messenger RNA (mRNA) or microRNA (miRNA)) template in a reaction catalyzed by reverse transcriptase.

Engineered nucleic acids of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. Other methods of producing engineered nucleic acids may be used in accordance with the present disclosure.

A gene is a distinct sequence of nucleotides, the order of which determines the order of monomers in a polynucleotide or polypeptide. A gene typically encodes a protein. A gene may be endogenous (occurring naturally in a host organism) or exogenous (transferred, naturally or through genetic engineering, to a host organism). An allele is one of two or more alternative forms of a gene that arise by mutation and are found at the same locus on a chromosome. A gene, in some embodiments, includes a promoter sequence, coding regions (e.g., exons), non-coding regions (e.g., introns), and regulatory regions (also referred to as regulatory sequences).

A mouse comprising a human gene is considered to comprise a human transgene. A transgene is a gene exogenous to a host organism. That is, a transgene is a gene that has been transferred, naturally or through genetic engineering, to a host organism. A transgene does not occur naturally in the host organism (the organism, e.g., mouse, comprising the transgene).

A promoter is a nucleotide sequence to which RNA polymerase binds to initial transcription (e.g., ATG). Promoters are typically located directly upstream from (at the 5′ end of) a transcription initiation site. In some embodiments, a promoter is an endogenous promoter. An endogenous promoter is a promoter that naturally occurs in that host animal.

An open reading frame is a continuous stretch of codons that begins with a start codon (e.g., ATG), ends with a stop codon (e.g., TAA, TAG, or TGA), and encodes a polypeptide, for example, a protein. An open reading frame is operably linked to a promoter if that promoter regulates transcription of the open reading frame.

An exon is a region of a gene that codes for amino acids. An intron (and other non-coding DNA) is a region of a gene that does not code for amino acids.

A nucleotide sequence encoding a product (e.g., protein), in some embodiments, has a length of 200 base pairs (bp) to 100 kilobases (kb). The nucleotide sequence, in some embodiments, has a length of at least 10 kb. For example, the nucleotide sequence may have a length of at least 15 kb, at least 20 kb, at least 25 kb, at least 30 kb, or at least 35 kb. In some embodiments, the nucleotide sequence has a length of 10 to 100 kb, 10 to 75 kb, 10 to 50 kb, 10 to 30 kb, 20 to 100 kb, 20 to 75 kb, 20 to 50 kb, 20 to 30 kb, 30 to 100 kb, 30 to 75 kb, or 30 to 50 kb.

Any one of the nucleic acids provided herein may have a length of 200 bp to 500 kb, 200 bp to 250 kb, or 200 bp to 100 kb. A nucleic acid, in some embodiments, has a length of at least 10 kb. For example, a nucleic acid may have a length of at least 15 kb, at least 20 kb, at least 25 kb, at least 30 kb, at least 35 kb, at least 50 kb, at least 100 kb, at least 200 kb, at least 300 kb, at least 400 kb, or at least 500 kb. In some embodiments, a nucleic acid has a length of 10 to 500 kb, 20 to 400 kb, 10 to 300 kb, 10 to 200 kb, or 10 to 100 kb. In some embodiments, a nucleic acid has a length of 10 to 100 kb, 10 to 75 kb, 10 to 50 kb, 10 to 30 kb, 20 to 100 kb, 20 to 75 kb, 20 to 50 kb, 20 to 30 kb, 30 to 100 kb, 30 to 75 kb, or 30 to 50 kb. A nucleic acid may be circular or linear.

The nucleic acids described herein, in some embodiments, include a modification. A modification, with respect to a nucleic acid, is any manipulation of the nucleic acid, relative to the corresponding wild-type nucleic acid (e.g., the naturally-occurring nucleic acid). A genomic modification is thus any manipulation of a nucleic acid in a genome (e.g., in a coding region, non-coding region, and/or regulatory region), relative to the corresponding wild-type nucleic acid (e.g., the naturally-occurring (unmodified) nucleic acid) in the genome. Non-limiting examples of nucleic acid (e.g., genomic) modifications include deletions, insertions, “indels” (deletion and insertion), and substitutions (e.g., point mutations). In some embodiments, a deletion, insertion, indel, or other modification in a gene results in a frameshift mutation such that the gene no longer encodes a functional product (e.g., protein). Modifications also include chemical modifications, for example, chemical modifications of at least one nucleobase. Methods of nucleic acid modification, for example, those that result in gene inactivation, are known and include, without limitation, RNA interference, chemical modification, and gene editing (e.g., using recombinases or other programmable nuclease systems, e.g., CRISPR/Cas, TALENs, and/or ZFNs).

A loss-of-function mutation, as is known in the art, results in a gene product with little or no function. A null mutation, which is a type of loss-of-function mutation, results in a gene product with no function. In some embodiments, an inactivated allele is a null allele. Other examples of loss-of-function mutations includes missense mutations and frameshift mutations.

A nucleic acid, such as an allele or alleles of a gene, may be modified such that it does not produce a detectable level of a functional gene product (e.g., a functional protein). Thus, an inactivated allele is an allele that does not produce a detectable level of a functional gene product (e.g., a functional protein). A detectable level of a protein is any level of protein detected using a standard protein detection assay, such as flow cytometry and/or an ELISA. In some embodiments, an inactivated allele is not transcribed. In some embodiments, an inactivated allele does not encode a functional protein.

Vectors used for delivery of a nucleic acid include minicircles, plasmids, bacterial artificial chromosomes (BACs), and yeast artificial chromosomes. It should be understood, however, that a vector may not be needed. For example, a circularized or linearized nucleic acid may be delivered to an embryo without its vector backbone. Vector backbones are small (˜4 kb), while donor DNA to be circularized can range from >100 bp to 50 kb, for example.

Methods for delivering nucleic acids to mouse embryos (e.g., mouse) for the production of transgenic mice include, but are not limited to, electroporation (see, e.g., Wang W et al. J Genet Genomics 2016; 43 (5): 319-27; WO 2016/054032; and WO 2017/124086, each of which is incorporated herein by reference), DNA microinjection (see, e.g., Gordon and Ruddle, Science 1981:214:1244-124, incorporated herein by reference), embryonic stem cell-mediated gene transfer (see, e.g., Gossler et al., Proc. Natl. Acad. Sci. 1986; 83:9065-9069, incorporated herein by reference), and retrovirus-mediated gene transfer (see, e.g., Jaenisch, Proc. Natl. Acad. Sci. 1976; 73:1260-1264, incorporated herein by reference), any of which may be used as provided herein.

### Genomic Editing Methods

The present application contemplates the use of a variety of gene editing technologies using engineered nucleic acids, for example, to knockout a target gene (e.g., Fcgr1) or to introduce nucleic acids into the genome of a mouse (e.g., to produce a transgenic mouse). An immunodeficient Fcgr1^{null} mouse may be produced by any gene editing technology known in the art. In some embodiments, an immunodeficient Fcgr1^{null} mouse further comprises a human interleukin-15 transgene (integrated into its genome).

Engineered nucleic acids, such as guide RNAs, donor polynucleotides, and other nucleic acid coding sequences, for example, may be introduced to a genome of an embryo or cell (e.g., stem cell) using any suitable method. The present application contemplates the use of a variety of gene editing technologies, for example, to delete nucleic acids from the genome of an embryo or cell to produce a knockout mouse or to introduce nucleic acids into the genome of an embryo or cell to produce a transgenic mouse. Non-limiting examples include programmable nuclease-based systems, such as clustered regularly interspaced short palindromic repeat (CRISPR) systems, zinc-finger nucleases (ZFNs), and transcription activator-like effector nucleases (TALENs). See, e.g., Carroll D Genetics. 2011; 188 (4): 773-782; Joung J K et al. Nat Rev Mol Cell Biol. 2013; 14 (1): 49-55; and Gaj T et al. Trends Biotechnol. 2013 July; 31 (7): 397-405, each of which is incorporated by reference herein.

In some embodiments, a CRISPR system is used to edit the genome of mouse (e.g., mouse) embryos provided herein. See, e.g., Harms D W et al., Curr Protoc Hum Genet. 2014; 83:15.7.1-15.7.27; and Inui M et al., Sci Rep. 2014; 4:5396, each of which are incorporated by reference herein). For example, Cas9 mRNA or protein, one or multiple guide RNAs (gRNAs), and/or a donor nucleic acid can be delivered, e.g., injected or electroporated, directly into mouse embryos at the one-cell (zygote) stage or a later stage to facilitate homology directed repair (HDR), for example, to delete a nucleic acid sequence from the genome or to introduce an engineered nucleic acid (e.g., donor nucleic acid) into the genome.

The CRISPR/Cas system is a naturally occurring defense mechanism in prokaryotes that has been repurposed as an RNA-guided-DNA-targeting platform for gene editing. Engineered CRISPR systems contain two main components: a guide RNA (gRNA) and a CRISPR-associated endonuclease (e.g., Cas protein). The gRNA is a short synthetic RNA composed of a scaffold sequence for nuclease-binding and a user-defined nucleotide spacer (e.g., ˜15-25 nucleotides, or ˜20 nucleotides) that defines the genomic target (e.g., gene) to be modified. Thus, one can change the genomic target of the Cas protein by simply changing the target sequence present in the gRNA. In some embodiments, the Cas9 endonuclease is from Streptococcus pyogenes (NGG PAM) or Staphylococcus aureus (NNGRRT or NNGRR (N) PAM), although other Cas9 homologs, orthologs, and/or variants (e.g., evolved versions of Cas9) may be used, as provided herein. Additional non-limiting examples of RNA-guided nucleases that may be used as provided herein include Cpf1 (TTN PAM); SpCas9 D1135E variant (NGG (reduced NAG binding) PAM); SpCas9 VRER variant (NGCG PAM); SpCas9 EQR variant (NGAG PAM); SpCas9 VQR variant (NGAN or NGNG PAM); Neisseria meningitidis (NM) Cas9 (NNNNGATT PAM); Streptococcus thermophilus (ST) Cas9 (NNAGAAW PAM); and Treponema denticola (TD) Cas9 (NAAAAC). In some embodiments, the CRISPR-associated endonuclease is selected from Cas9, Cpf1, C2c1, and C2c3. In some embodiments, the Cas nuclease is Cas9.

A guide RNA comprises at least a spacer sequence that hybridizes to (binds to) a target nucleic acid sequence and a CRISPR repeat sequence that binds the endonuclease and guides the endonuclease to the target nucleic acid sequence. As is understood by the person of ordinary skill in the art, each gRNA is designed to include a spacer sequence complementary to its genomic target sequence. See, e.g., Jinek et al., Science, 2012; 337:816-821 and Deltcheva et al., Nature, 2011; 471:602-607, each of which is incorporated by reference herein.

In some embodiments, the RNA-guided nuclease and the gRNA are complexed to form a ribonucleoprotein (RNP), prior to delivery to an embryo.

The concentration of RNA-guided nuclease or nucleic acid encoding the RNA-guided nuclease may vary. In some embodiments, the concentration is 100 ng/μl to 1000 ng/μl. For example, the concentration may be 100, 150, 200, 250, 300, 350, 400, 450, 500, 550, 600, 650, 700, 750, 800, 850, 900, 950, or 1000 ng/μl. In some embodiments, the concentration is 100 ng/μl to 500 ng/μl, or 200 ng/μl to 500 ng/μl.

The concentration of gRNA may also vary. In some embodiments, the concentration is 200 ng/μl to 2000 ng/μl. For example, the concentration may be 200, 300, 400, 500, 600, 700, 800, 900, 1000, 1100, 1200, 1300, 1400, 1500, 1600, 1700, 1700, 1900, or 2000 ng/μl. In some embodiments, the concentration is 500 ng/μl to 1000 ng/μl. In some embodiments, the concentration is 100 ng/μl to 1000 ng/μl. For example, the concentration may be 100, 150, 200, 250, 300, 350, 400, 450, 500, 550, 600, 650, 700, 750, 800, 850, 900, 950, or 1000 ng/μl.

In some embodiments, the ratio of concentration of RNA-guided nuclease or nucleic acid encoding the RNA-guided nuclease to the concentration of gRNA is 2:1. In other embodiments, the ratio of concentration of RNA-guided nuclease or nucleic acid encoding the RNA-guided nuclease to the concentration of gRNA is 1:1.

A donor nucleic acid typically includes a sequence of interest flanked by homology arms. Homology arms are regions of the ssDNA that are homologous to regions of genomic DNA located in a genomic locus. One homology arm is located to the left (5′) of a genomic region of interest (into which a sequence of interest is introduced) (the left homology arm) and another homology arm is located to the right (3′) of the genomic region of interest (the right homology arm). These homology arms enable homologous recombination between the ssDNA donor and the genomic locus, resulting in insertion of the sequence of interest into the genomic locus of interest (e.g., via CRISPR/Cas9-mediated homology directed repair (HDR)).

The homology arms may vary in length. For example, each homology arm (the left arm and the right homology arm) may have a length of 20 nucleotide bases to 1000 nucleotide bases. In some embodiments, each homology arm has a length of 20 to 200, 20 to 300, 20 to 400, 20 to 500, 20 to 600, 20 to 700, 20 to 800, or 20 to 900 nucleotide bases. In some embodiments, each homology arm has a length of 20, 30, 40, 50, 60, 70, 80, 90, 100, 150, 200, 250, 300, 350, 400, 450, 500, 550, 600, 650, 700, 750, 800, 850, 900, 950, or 1000 nucleotide bases. In some embodiments, the length of one homology arm differs from the length of the other homology arm. For example, one homology arm may have a length of 20 nucleotide bases, and the other homology arm may have a length of 50 nucleotide bases. In some embodiments, the donor DNA is single stranded. In some embodiments, the donor DNA is double stranded. In some embodiments, the donor DNA is modified, e.g., via phosphorothioation. Other modifications may be made.

### EXAMPLES

### Example 1. Producing an NSG-Fcgr1^{null} and NSG-Fcgr1^{null} Tg(Hu-IL15) Mice

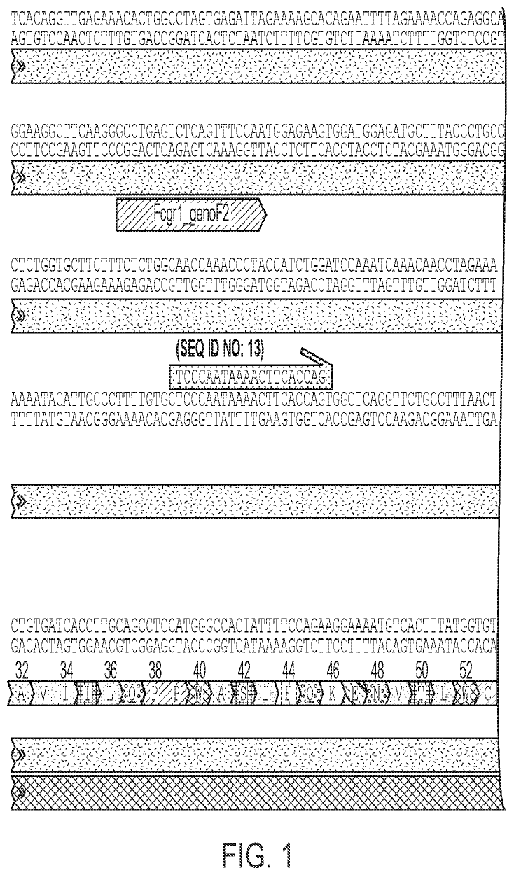

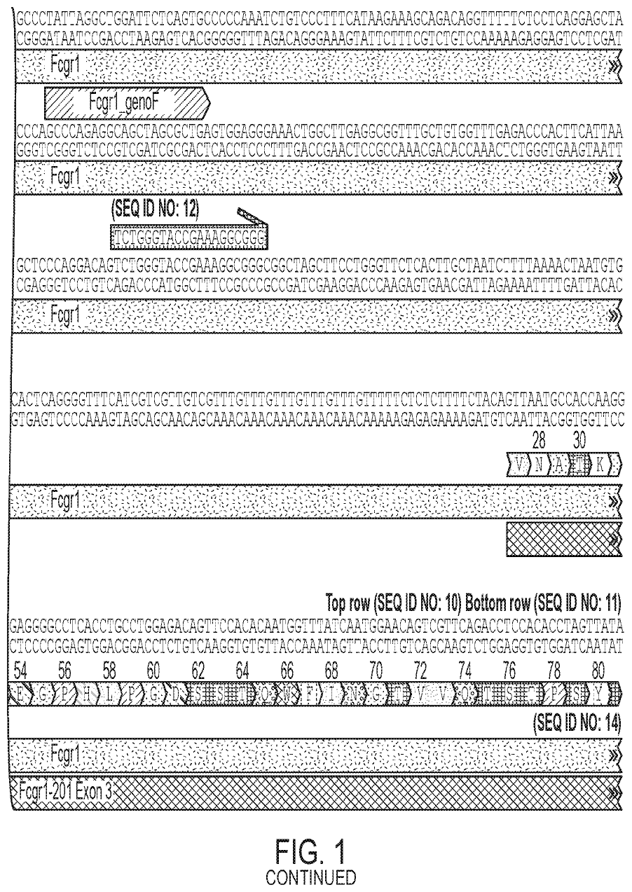

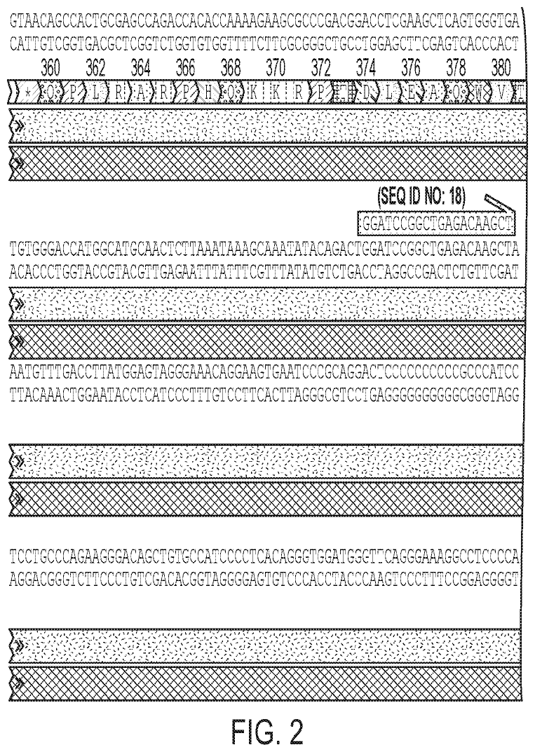

CRISPR-Cas9 genomic editing was used to delete exons 3-6 from the mouse Fcgr1 gene in NSG™ mice (JAX Strain No.: 005557) and NSG™ mice transgenically expressing human IL15 (NSG-Tg(Hu-IL15)) (JAX Strain No.: 030890). Deletion of exons 3-6 is expected to produce a ˜8500 base pair deletion. The mouse Fcgr1 gene locus encodes a 404 amino acid protein coding transcript (Fcgr1-201) and a 50 amino acid nonsense mediated decay transcript (Fcgr1-202). CRISPR-Cas9 gene primers were designed upstream (FIG. 1) and downstream (FIG. 2) of the targeted exons 3-6. See Table 1 below.

TABLE 1
CRISPS-Cas9 Genomic Editing Targeting Strategy to Delete Exons 3-6 of the
Fcgr1 Gene NSG ™ and in NSG-Tg(Hu-IL15 Mice)

| Gene | Name | Fcgrl (Fc receptor, IgG, high affinity I) |
|---|---|---|
| | Location | Chr3:96282909-96293969 bp, − strand |
| | Transcript | ENSMUST00000029748.7 |
| | ID | |
| Model Desired | | Deletion of exons 3-6 (8495 bp deletion) |
| Parental Strain | | NOD.Cg-Prkdc^{scid} Il2rg^{tm1Wj1}/SzJ (JAX Strain #: 00555) |
| CRISPR | Guides | Fcgr1_up_crRNA1: TCTGGGTACCGAAAGGCGGG (SEQ ID NO: 2) |
| | | Fcgr1_up_crRNA2: TCCCAATAAAACTTCACCAG (SEQ ID NO: 3) |
| | | Fcgrl_down_crRNA1: GGATCCGGCTGAGACAAGCT (SEQ ID NO: 4) |
| | | Fcgrl_down_crRNA2: ATGGAGTCAGGTCACAGCGG (SEQ ID NO: 5) |

The off-target genomic editing risk for the CRISPR-Cas9 primers was evaluated. When the CRISPR-Cas9 primers have a linked, non-canonical protospacer adjacent motif (PAM), the off-target genomic editing risk is considered high when the score is <20. All 4 primers have minimal off-target genomic editing risk because they have a linked, non-canonical and their off-target editing risk score is <3.5.

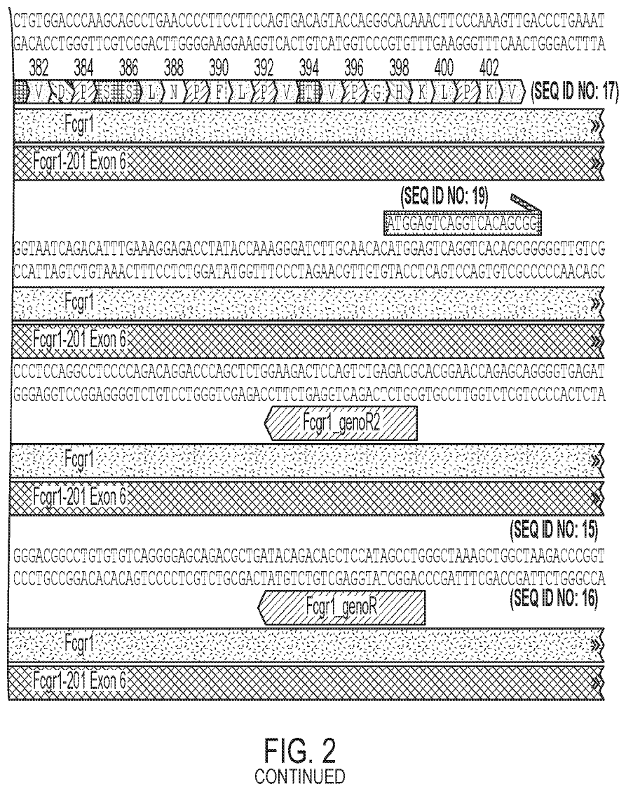

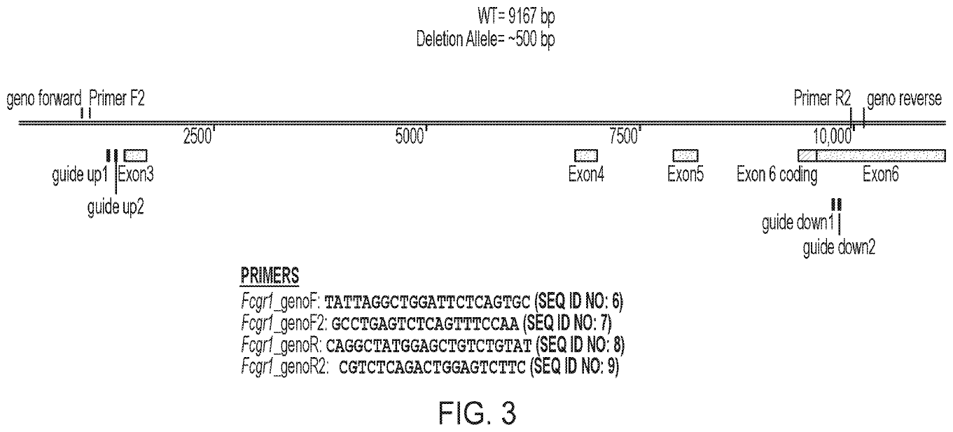

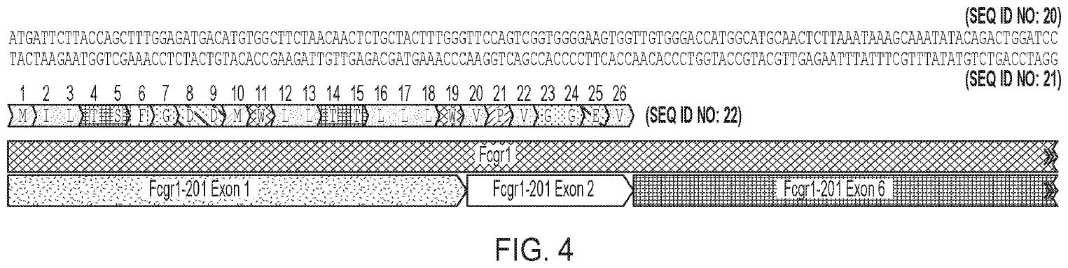

Deletion of mouse Fcgr1 exons 3-6 is expected to produce a mutant allele, which may be detected by sequencing beginning upstream of targeted exon 3 in the forward direction and/or downstream of exon 6 is the reverse direction (FIG. 3). This deletion is also expected to produce an early truncation of the Fcgr1-201 protein coding transcript after amino acid 26 (FIG. 4).

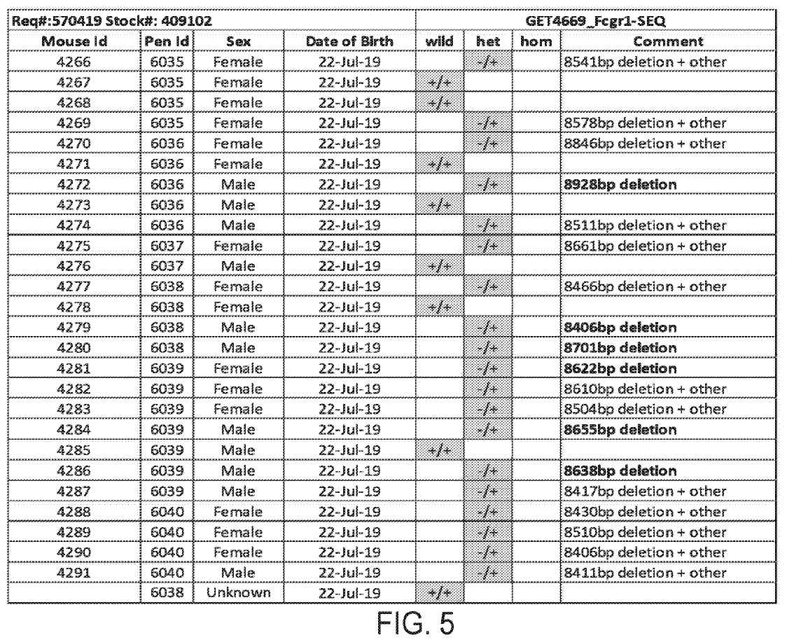

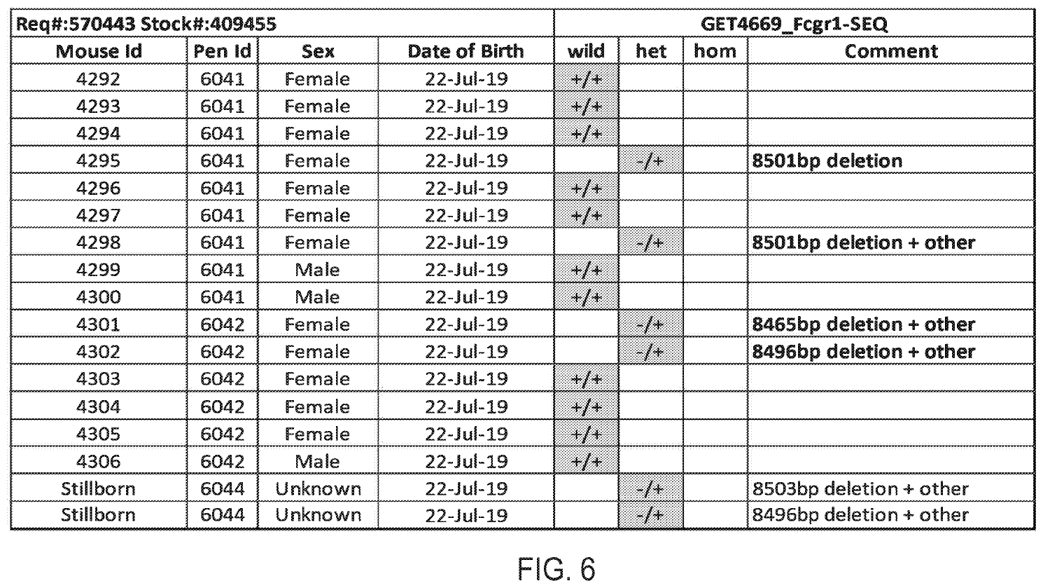

Nineteen founder NSG™ mice heterozygous for the Fcgr1 deletion of exons 3-6 (Fcgr1^{null}) were produced (FIG. 5). Six founder NSG-Tg(Hu-IL15) mice heterozygous for the Fcgr1 deletion of exons 3-6 (Fcgr1^{null}) were produced (FIG. 6).

### Example 2. Decreased Clearance of Human IgG1 Antibody in NSG-Fcgr1^{null} Mice

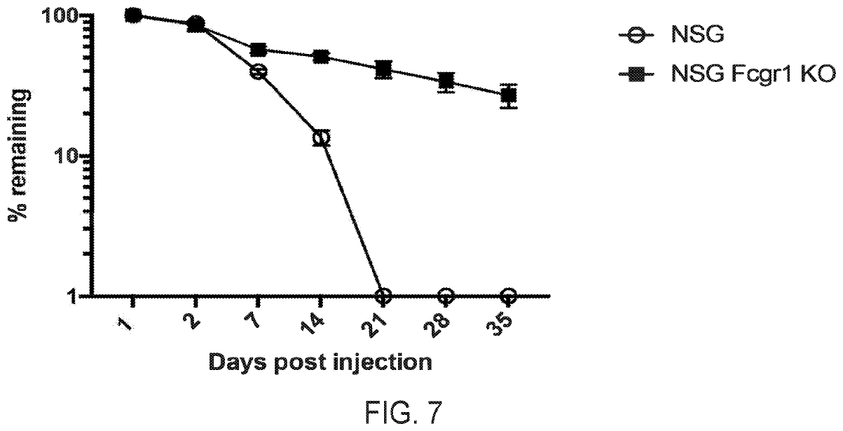

The clearance of the therapeutic human immunoglobulin 1 (IgG1) antibody trastuzumab (Herceptin®) was compared in NSG™ and NSG-Fcgr1^{null} mice. Trastuzumab is approved for early-stage breast cancer that is human epidermal growth factor R2-positive (HER2+). Groups of five NSG-Fcgr1^{null} and five NSG™ control mice were injected intravenously with 200 μg of trastuzumab. In 25 g mice, this is a dose of ˜8 mg/kg. Mice were bled and sera was frozen at 1, 2, 7, 14, 21, 28, and 35 days post-trastuzumab injection. The percentage of initial trastuzumab dose remaining was determined by enzyme-linked immunosorbent assay (ELISA). While trastuzumab was rapidly cleared in NSG™ mice (1% remaining at day 21 post-injection), there was decreased clearance and increased persistence of trastuzumab in NSG-Fcgr1^{null} mice (˜40% remaining at day 35 post-injection) (FIG. 7).

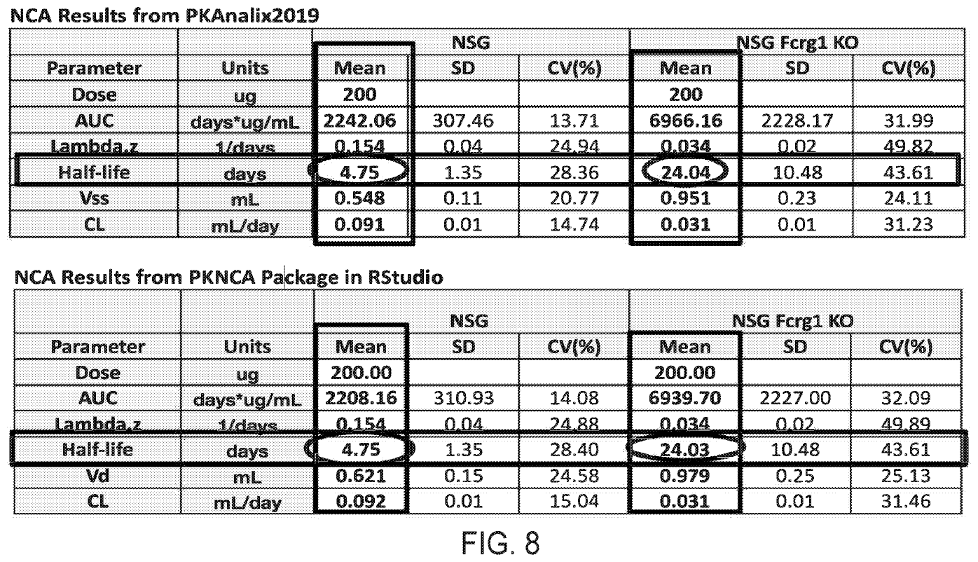

Two different programs (PKAnalix2019 and R_Studio) were used calculate the pharmacokinetics of trastuzumab clearance in NSG™ and NSG-Fcgr1^{null} mice. The half-life of trastuzumab in NSG-Fcgr1^{null} mice is approximately 24 days compared with approximately 5 days in NSG™ mice (FIG. 8). Thus, knocking out the Fcgr1 gene in NSG™ mice resulted in a 5-fold increase in the half-life of trastuzumab in circulation. This increased half-life is similar to the half-life of trastuzumab in humans (Leveque et al., Curr Clin Pharmacol., (2008), 3 (1): 51-55).

### Example 3. Decreased Clearance of Human IgG4 Antibody in NSG-Fcgr1^{null} Mice

One of the most efficient approaches for activation of anti-tumor immunity is the blockade of immune checkpoints. Tumors use immune checkpoints to suppress anti-tumor immune responses. Blockade of immune checkpoint proteins, such as programmed cell death protein 1 (PD-1), has presented broad and diverse opportunities to enhance anti-tumor immunity with the potential to produce durable clinical responses. Pembrolizumab (Keytruda®) is one of the most widely-used immune checkpoint blockade monoclonal antibodies (mAbs). Pembrolizumab is a humanized IgG4-kappa mAb that recognizes human PD-1. PD-1 is broadly expressed on activated CD4⁺ T cells, CD8⁺ T cells, activated CD4⁺ regulatory T (T_{reg}) cells, B cells, and natural killer (NK) cells. PD-1 is also constitutively expressed on tumor-infiltrating lymphocytes (TILs) in a variety of tumor types, reflecting an exhausted T-cell status. PD-1 binds to two ligands: PD-1 ligand 1 (PD-LI; also known as B7-H1) and PD-L2 (B7-DC). PD-L1 is broadly expressed on healthy (non-cancerous) cells and malignant (cancerous) cells, and PD-L2 is predominantly expressed on antigen presenting cells. PD-L1 binding to PD-1 leads to inhibition of T-cell activation and effector function mediated by recruitment of tyrosine phosphatases to the immune synapse that disrupts T-cell receptor signaling. A large body of evidence has demonstrated that PD-L1 expression is commonly upregulated in many different human cancer types, including melanoma, lung, and ovarian tumors.

Engraftment of NSG™ mice with human tumor cell lines and patient-derived tumor xenografts followed by treatment with multiple injections of the human IgG4 therapeutic antibody pembrolizumab (Keytruda®) at 5-10 mg/kg every 5 days results in slower tumor growth in some, but not all experiments. The reduced effect of pembrolizumab on human tumor therapy in NSG™ mice may be associated with rapid clearance of this human IgG4 antibody due to increased activity of the mouse Fcgr1 receptor.

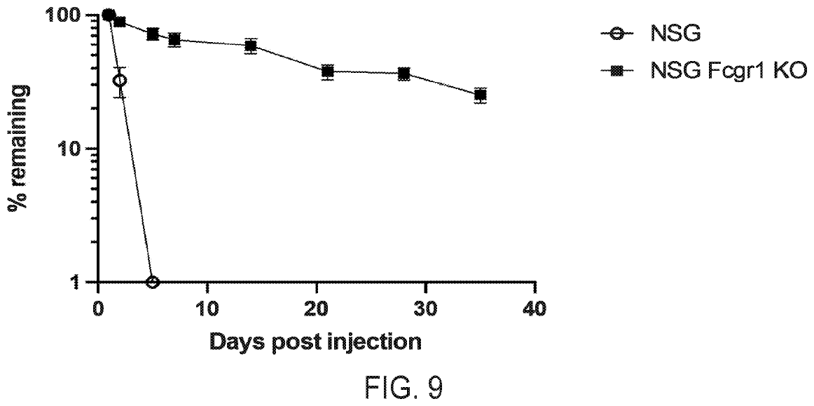

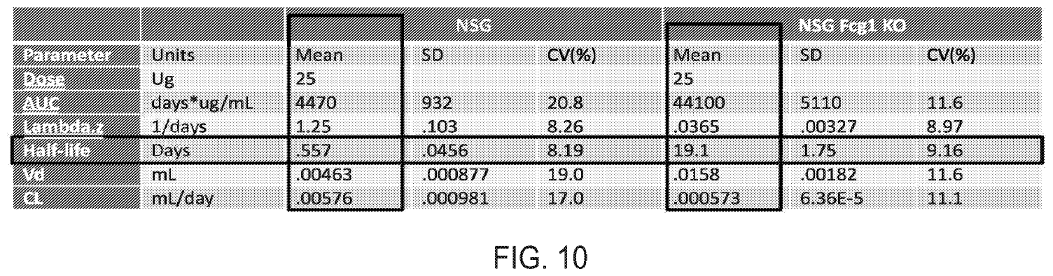

To test this hypothesis, clearance of pembrolizumab was compared in NSG™ and NSG-Fcgr1^{null} mice. Groups of five NSG-Fcgr1^{null} and five NSG™ control mice were injected intravenously with 25 μg of pembrolizumab. In 25 g mice, this is a dose of ˜1 mg/kg. Mice were bled and sera was frozen at 1-35 days post-pembrolizumab injection. The percentage of initial pembrolizumab dose remaining was determined by enzyme-linked immunosorbent assay (ELISA). While pembrolizumab was rapidly cleared in NSG™ mice (>1% remaining by day 6 post-injection), there was decreased clearance and increased persistence of pembrolizumab in NSG-Fcgr1^{null} mice (˜30% remaining at day 35 post-injection) (FIG. 9).

Two different programs (PKAnalix2019 and R_Studio) were used calculate the pharmacokinetics of pembrolizumab clearance in NSG™ and NSG-Fcgr1^{null} mice. The half-life of pembrolizumab in NSG-Fcgr1^{null} mice is approximately 19 days compared with only approximately 0.6 days in NSGTMmice (FIG. 10). Thus, knocking out the Fcgr1 gene in NSG™ mice resulted in a 30-fold increase in the half-life of pembrolizumab in circulation. This increased half-life is similar to the half-life of pembrolizumab in humans (Centanni et al., Clinical Pharmacokinetics, (2019), 58:85-857).

### Example 4. Increased Circulating Human IgG Antibody Levels in NSG-Fcgr1^{null} Mice

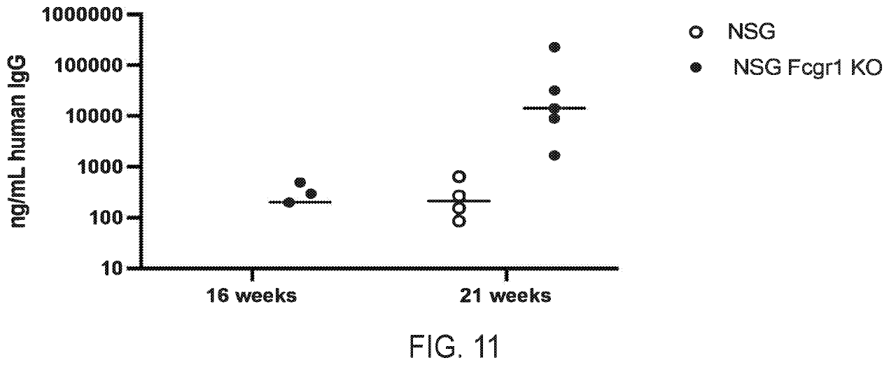

Attempts to generate human IgG antibodies following immunization of NSG™ mice engrafted with human CD34+ hematopoietic stem cells (HSCs) have been limited by the short half-life of the circulating human IgG. NSG™ and NSG-Fcgr1^{null} mice were engrafted with human umbilical cord HSCs, and the level of human IgG was measured in plasma at 21 weeks post-engraftment. The NSG-Fcgr1^{null} mice had 20-fold higher levels of circulating human IgG compared to NSG™ mice (FIG. 11). This is consistent with increased persistence of human IgG therapeutic antibodies (trastuzumab and pembrolizumab) in the circulation of NSG-Fcgr1^{null} mice compared to NSG™ mice.

All references, patents and patent applications disclosed herein are incorporated by reference with respect to the subject matter for which each is cited, which in some cases may encompass the entirety of the document.

The indefinite articles “a” and “an,” as used herein the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

1. An immunodeficient mouse comprising a mouse Fc gamma receptor 1 null (Fcgr1^{null}) allele and a mouse interleukin-2 receptor gamma null (IL-2Rγ^{null}) allele.

2. The immunodeficient mouse of claim 1, wherein the mouse comprises a non-obese diabetic (NOD) genetic background.

3. The immunodeficient mouse of claim 1 or 2, wherein the mouse is homozygous for the IL-2Rγ^{null} allele and is homozygous for the Fcgr1^{null} allele.

4. The immunodeficient mouse of any one of claims 1-3, wherein the mouse further comprises Prkdc^{scid} allele.

5. The immunodeficient mouse of claim 4, wherein the mouse is homozygous for the Prkdc^{scid} allele.

6. The immunodeficient mouse of any one of claims 1-3, wherein the mouse further comprises a Rag1^{null} allele.

7. The immunodeficient mouse of claim 6, wherein the mouse is homozygous for the Rag1^{null} allele.

8. The immunodeficient mouse of any one of the preceding claims, wherein the mouse lacks mouse T cells, B cells, and/or natural killer (NK) cells.

9. The immunodeficient mouse of any one of the preceding claims, wherein macrophage and/or dendritic cell function in the mouse is defective.

10. The immunodeficient mouse of any one of the preceding claims, engrafted with human hematopoietic stem cells (HSCs).

11. The immunodeficient mouse of any one of the preceding claims, engrafted with human peripheral blood mononuclear cells (PBMCs).

12. The immunodeficient mouse of any one of the preceding claims, engrafted with human diseased cells.

13. The immunodeficient mouse of claim 12, wherein the human diseased cells are human tumor cells.

14. The immunodeficient mouse of claim 12, wherein the human tumor cells are human cancer cells.

15. The immunodeficient mouse of any one of the preceding claims, wherein the mouse Fcgr1^{null} allele comprises a deletion in a region of exons 3-6, relative to an endogenous unmodified mouse Fcgr1 gene.

16. The immunodeficient mouse of claim 15, wherein the mouse Fcgr1^{null} allele comprises a deletion of exons 3-6.

17. The immunodeficient mouse of any one of the preceding claims, further comprising a human IL-15 transgene.

19. The immunodeficient mouse of claim 17, wherein the human IL-15 transgene is integrated into the genome of the mouse.

19. A method comprising administering an antibody to the immunodeficient mouse of any one of the preceding claims.

20. The method of claim 19, wherein the antibody is a monoclonal antibody.

21. The method of claim 19 or 20, wherein the antibody is a human or humanized antibody.

22. The method of any one of the preceding claims, wherein the antibody is an IgG1, IgG2, IgG3 or IgG4 antibody.

23. The method of any one of the preceding claims, wherein the antibody is an immune checkpoint inhibitor (ICI) antibody.

24. The method of claim 23, wherein the ICI antibody is selected from anti-PD-1 antibodies, anti-PD-L1 antibodies, and anti-CTLA-4 antibodies.

25. The method of any one of the preceding claims, further comprising assaying a biological sample from the mouse for a therapeutic effect of the antibody.

26. The method of claim 25, wherein the therapeutic effect is decreased growth of human diseased cells, relative to a control.

27. The method of any one of the preceding claims, further comprising assaying a biological sample from the mouse for a circulating level of the antibody.

28. The method of claim 27, wherein the circulating level of the antibody is at least 5-fold, at least 10-fold, at least 15-fold, or at least 20-fold higher, relative to a control.

29. The method of any one of claims 25-28, wherein the assaying is at least 21, at least 28, or at least 35 days post-administration of the antibody.

30. A method comprising administering human cells to the immunodeficient mouse of any one of claim 1-9.

31. The method of claim 30, wherein the human cells are human hematopoietic stem cells (HSCs).

32. The method of claim 30, wherein the human cells are human peripheral blood mononuclear cells (PBMCs).

33. The method of any one of claims 30-32, wherein the human cells are human diseased cells.

34. The method of claim 33, wherein the human diseased cells are human tumor cells.

35. The method of claim 34, wherein the human tumor cells are human cancer cells.

36. An immunodeficient mouse comprising a mouse Fc gamma receptor 1 null (Fcgr1^{null}) allele, wherein the mouse comprises an NOD.Cg-Prkdc^{scid}Il2rg^{tm1Wj1}/SzJ background.

37. The immunodeficient mouse of claim 36, wherein the mouse is homozygous for the Fcgr1^{null} allele.

38. The immunodeficient mouse of claim 36 or 37, wherein the mouse Fcgr1^{null} allele comprises a deletion in a region of exons 3-6, relative to an endogenous unmodified mouse Fcgr1 gene.

39. The immunodeficient mouse of claim 38, wherein the mouse Fcgr1^{null} allele comprises a deletion of exons 3-5.

40. The immunodeficient mouse of any one of claims 36-39, further comprising a human IL-15 transgene.

41. The immunodeficient mouse of claim 40, wherein the human IL-15 transgene is integrated into the genome of the mouse.

42. The immunodeficient mouse of any one of claims 36-41, engrafted with human hematopoietic stem cells (HSCs).

43. The immunodeficient mouse of any one of claims 36-41, engrafted with human peripheral blood mononuclear cells (PBMCs).

44. The immunodeficient mouse of any one of claims 36-43, engrafted with human diseased cells.

45. The immunodeficient mouse of claim 44, wherein the human diseased cells are human tumor cells.

46. The immunodeficient mouse of claim 45, wherein the human tumor cells are human cancer cells.

47. A guide RNA comprising the sequence of any one of SEQ ID NOs: 2-5.

48. A method of producing the immunodeficient mouse of any one of the preceding claims comprising introducing one or more guide RNA and a Cas protein into a mouse embryo; transplanting the mouse embryo into a pseudopregnant female mouse; collecting F1 mice born from the pseudopregnant female; and breeding the F1 mice.

49. The method of claim 48, wherein the one or more guide RNA binds to a region upstream from exon 3 and/or downstream from exon 6 of the mouse Fcgr1 gene, optionally a guide RNA of claim 48.

50. The method of claim 48 or 49, wherein the mouse embryo is an immunodeficient mouse embryo, optionally having an NOD genetic background, and optionally comprising one or more of (a) an IL-2Rγ^{null} allele and (b) an Prkdc^{scid} allele or a Rag1^{null} allele.
